(19) United States
(12) Patent Application Publication
(10) Pub. No.: US 2026/0348034 A1
(43) Pub. Date: Nov. 5, 2026

(54) SYSTEMS AND METHODS FOR MONITORING ELECTROCHEMICAL SYNTHESIS

(71) Applicant: Atlas Data Storage, Inc., South San Francisco, CA (US)

(72) Inventors: Pranav SUNDARAM, South San Francisco, CA (US); Tom STERN, South San Francisco, CA (US); Dominique TOPPANI, South San Francisco, CA (US); Roger RUDOFF, South San Francisco, CA (US)

(21) Appl. No.: 19/662,341

(22) Filed: Apr. 29, 2026

Related U.S. Application Data
(60) Provisional application No. 63/797,740, filed on Apr. 30, 2025.

(51) Int. Cl. C25B 15/025; C07H 1/00; C07H 21/00

(52) U.S. Cl. CPC C25B 15/025; C07H 1/00; C07H 21/00

(57) ABSTRACT

Described herein are systems and methods including conducting a biomolecular synthesis reaction on a primary array, exposing a secondary array to a secondary inert redox probe, wherein the secondary inert redox probe provides data on the electrochemical chip condition, and wherein the secondary array is connected to a potentiostat; receiving data regarding the secondary array, comparing a feature of the data to a pre-determined threshold, and based on the comparison determining a condition of the electrochemical chip. Also described is a system for monitoring electrochemical synthesis, including an electrochemical chip including a primary array, a secondary array, and a secondary inert redox probe exposed to the secondary array, wherein the system is configured to receive data regarding a property of the secondary inert redox probe, compare the data to a pre-determined threshold, and based on the comparison, determine a condition of the electrochemical chip.

### CROSS-REFERENCE TO RELATED APPLICATION

This application claims priority to and the benefit of U.S. Provisional Patent Application No. 63/797,740, filed Apr. 30, 2025, and titled “SYSTEMS AND METHODS FOR MONITORING ELECTROCHEMICAL SYNTHESIS,” the disclosure of which is hereby incorporated by reference herein in its entirety.

### BACKGROUND

Electrochemical synthesis of oligomers, peptides and other biomolecules on an electrode array can include cyclic addition of monomers onto a growing chain and deblocking the previous monomer. Deblocking the previous monomer can include applying an electrical potential to generate a chemical moiety that participates in the deblocking reaction. During directed electrochemical synthesis that includes deblocking, monitoring and controlling sub-processes during synthesis can be important to the efficacy of the synthesis. Sub-processes may include electrode fouling or degradation, side product generation, reagent degradation, and other chemical/fluidic effects that can affect synthesis quality.

### SUMMARY

Embodiments of the present disclosure include methods of determining a condition on an electrochemical chip including a plurality of reaction sites. The method may include conducting a biomolecular synthesis reaction on a primary array of the plurality of reaction sites; exposing a secondary array of the plurality of reaction sites to a secondary inert redox probe, wherein the secondary inert redox probe provides data on the electrochemical chip condition, and wherein the secondary array is connected to a potentiostat; receiving data regarding the secondary array, by a computing device; comparing a feature of the data to a pre-determined threshold, by a computing device; based on the comparison of the feature to the pre-determined threshold, determining, by the computing device, a condition of the electrochemical chip; and generating, by the computing device, a signal indicative of the condition of the electrochemical chip.

Embodiments of the methods described herein may further include altering an electrochemical synthesis parameter of the biomolecular synthesis, based on the determined condition. In addition or alternatively, methods described herein may further include synthesizing biomolecules on the secondary array. The data regarding the secondary array may be first data. In some embodiments, the methods may further include contacting the plurality of reaction sites with a washing solution; receiving, by a computing device, second data regarding the secondary array; and based on a comparison of the first data to the second data, determining, by the computing device, a cleaning state of one or more reaction sites of the plurality of reaction sites, and generating, by the computing device, a signal indicative of the cleaning state. The electrochemical synthesis parameter may include voltage, reactant concentration, and/or time. The data received regarding the secondary array may include an activity or a property of the secondary inert redox probe measured by the potentiostat. The secondary inert redox probe may include ferrocene. The received data may include outputs from cyclic voltammetry, chronoamperometry, chronocoulometry, and/or electrochemical impedance spectroscopy. The biomolecular synthesis may include oligonucleotide synthesis. Determining the condition of the electrochemical chip by the computing device may include assessing electrode quality, effectiveness of charge transfer, and/or adsorption. Assessing electrode quality may include measuring a thickness of an oxide layer, measuring a fouling of an electrode, measuring a corrosion of an electrode, and/or measuring an effective electrode area. The primary array and the secondary array may be exposed to identical electrochemical reactant concentrations and/or substantially the same voltage.

Embodiments of the present disclosure include methods of determining a condition on an electrochemical chip including a plurality of reaction sites. The method may include conducting a biomolecular synthesis reaction on a primary array of the plurality of reaction sites; connecting a secondary array of the plurality of reaction sites to a potentiostat; receiving data regarding the secondary array by a computing device; comparing a feature of the data to a pre-determined threshold by the computing device; and based on the comparison of the feature to the pre-determined threshold, determining, by the computing device, a condition of the electrochemical chip.

Embodiments of the methods described herein may further include altering an electrochemical synthesis parameter. The electrochemical synthesis parameter may include voltage, reactant concentration, depassivation, electrode cleaning, and/or time. In some embodiments, the data may include outputs from cyclic voltammetry, chronoamperometry, chronocoulometry, and/or electrochemical impedance spectroscopy. The biomolecular synthesis may include oligonucleotide synthesis. In addition or alternatively, methods described herein may further include synthesizing biomolecules on the secondary array. Determining the condition of the electrochemical chip may include assessing electrode quality, effectiveness of charge transfer, and/or adsorption. The secondary array may be located on a lid of the electrochemical chip. The secondary array may be exposed to a primary deblocking solution. Further provided herein are methods further used to select a primary deblocking solution for use during an electrochemical reaction, including contacting the plurality of reaction sites with a first primary deblocking solution; receiving first data regarding the secondary array; contacting the plurality of reaction sites with a second primary deblocking solution; receiving second data regarding the secondary array; comparing a feature of the first data to a feature of the second data; and selecting a primary deblocking solution based on the comparison of the feature of the first data to the feature of the second data. The contacting, receiving, and comparing may be repeated for two or more primary deblocking solutions. In some embodiments, the condition of the electrochemical chip may be determined during biomolecular synthesis. In other embodiments, the condition of the electrochemical chip may be determined before biomolecular synthesis. In addition or alternatively, the condition of the electrochemical chip may be determined after biomolecular synthesis.

In some embodiments, a system for monitoring electrochemical synthesis includes an electrochemical chip including a primary array configured for biomolecular synthesis and a secondary array configured for electrochemical monitoring, each of the primary array and the secondary array configured to receive the same chemical reagents, and the secondary array configured to be exposed to a secondary inert redox probe. The computing device is configured to receive data including a property of the secondary inert redox probe, compare the data to a pre-determined threshold, based on the comparison, determine a condition of the electrochemical chip, and generate a signal indicative of the determined condition.

### BRIEF DESCRIPTION OF THE DRAWINGS

Embodiments of the present disclosure may be implemented in connection with aspects illustrated in the attached drawings. These drawings show different aspects of the present disclosure, and where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. For simplicity and clarity of illustration, the figures depict the general structure and/or manner of construction of the various embodiments/aspects described herein.

FIG. 1A depicts a view of an exemplary electrochemical chip, according to some aspects of the present disclosure.

FIG. 1B depicts a block of a plurality of blocks on the exemplary electrochemical chip in FIG. 1A, according to some aspects of the present disclosure.

FIG. 1C depicts a reaction site in the block shown in FIG. 1B, according to some aspects of the present disclosure.

FIG. 2 depicts an exploded view of an exemplary flow cell assembly, including a flow cell top, the electrochemical chip as shown in FIGS. 1A-1C, and a flow cell bottom, according to some aspects of the present disclosure.

FIG. 3 depicts the exemplary flow cell assembly of FIG. 2 in a fully-assembled state, according to some aspects of the present disclosure.

FIG. 4A depicts a front view of a flow cell top of an exemplary flow cell assembly, according to some aspects of the present disclosure.

FIG. 4B depicts a rear view of a flow cell top of an exemplary flow cell assembly, according to some aspects of the present disclosure.

FIG. 4C depicts a front view of a flow cell bottom of an exemplary flow cell assembly, according to some aspects of the present disclosure.

FIG. 4D depicts a rear view of a flow cell bottom of an exemplary flow cell assembly, according to some aspects of the present disclosure.

FIG. 5 depicts an exemplary flow cell, according to some aspects of the present disclosure.

FIG. 6 depicts an exemplary fluid delivery pathway within a flow cell assembly, according to some aspects of the present disclosure.

FIG. 7A is a schematic representation of an electrical circuit for charge transfer from an electrode to a solution, according to some aspects of the present disclosure.

FIG. 7B is a schematic representation of an electrical circuit for charge transfer from an electrode to a solution with a parallel passivation resistor, according to some aspects of the present disclosure.

FIG. 8A depicts an exemplary electrochemical chip illustrating a presence of primary and secondary arrays, according to some aspects of the present disclosure.

FIG. 8B depicts an exemplary electrochemical chip illustrating different ways of arranging secondary arrays on an active area of an electrochemical chip, according to some aspects of the present disclosure.

FIG. 9A depicts an exemplary reaction site on a primary array with two electrodes, according to some aspects of the current disclosure.

FIG. 9B depicts an exemplary reaction site on a secondary array with three electrodes, according to some aspects of the current disclosure.

FIGS. 10A and 10B depict different variations of a secondary array, according to some aspects of the current disclosure.

FIG. 11 depicts an exemplary workflow of a method to determine a condition of an electrochemical chip, according to some aspects of the current disclosure.

FIG. 12 is a graph depicting an exemplary cyclic voltammogram (CV) (potential vs. Current curve) for a secondary inert redox solution on a 3-electrode configuration on a secondary array during synthesis of oligomers on the primary array, according to some aspects of the current disclosure.

FIG. 13 is a graph depicting the CV curve of a primary deblocking solution in a 3-electrode configuration on a secondary array, according to some aspects of the current disclosure.

FIG. 14 is a graph depicting a secondary inert redox probe CVs on a secondary array before every cycle of oligomer chemical synthesis, according to some aspects of the current disclosure.

FIG. 15 is a graph depicting a decrease in an absolute peak current for oxidation with cycling, according to some aspects of the current disclosure.

FIG. 16 is a graph depicting the secondary inert redox probe CV curve before and after the electrochemical deblocking step on a secondary array for 1 cycle, according to some aspects of the current disclosure.

FIG. 17 is a graph depicting the CV curve for the deblocking solution for 20 synthesis cycles for a 3-electrode configuration of a secondary array, according to some aspects of the current disclosure.

FIG. 18 is a graph depicting an example of using cyclic voltammetry on the secondary inert redox probe solution on a secondary array before and after cleaning, according to some aspects of the current disclosure.

FIG. 19 is a graph depicting an example of using cyclic voltammetry on the secondary inert redox probe solution on a secondary array after cleaning with three different cleaning methods.

FIG. 20 is a graph depicting a CV curve of an electrochemical deblocking solution, according to some aspects of the current disclosure.

FIG. 21 is a graph depicting a CV curve of an electrochemical deblocking solution, according to some aspects of the current disclosure.

FIG. 22 is a graph depicting a CV curve of an electrochemical deblocking solution, according to some aspects of the current disclosure.

FIG. 23 is a graph depicting the decrease in the absolute peak current for oxidation with cycling, according to some aspects of the current disclosure.

### DETAILED DESCRIPTION

There are many embodiments described and illustrated herein. The present disclosure is neither limited to any single aspect nor embodiment thereof, nor to any combinations and/or permutations of such aspects and/or embodiments. Moreover, each aspect of the present disclosure, and/or embodiments thereof, may be employed alone or in combination with one or more other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate that the embodiment(s) is/are “example” embodiment(s).

As used herein, the term “approximately” is meant to account for variations including those due to experimental error. When applied to numeric values, the terms “about” and “approximately” may indicate a variation of +/−5% from the disclosed numeric value, unless a different variation is specified. When applied to angle measures, the terms “about” and “approximately” may indicate a variation of +/−3°. As used herein, the singular forms “a,” “an,” and “the” include plural referents unless the context clearly dictates otherwise.

Throughout this disclosure, numerical features may be presented in a range format. It should be understood that the description in range format is merely for convenience and brevity, and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

### Definitions

Unless defined otherwise, each technical and scientific term used herein has the same meaning as is commonly understood by one of ordinary skill in the art to which these inventions belong.

As used herein, the terms “oligonucleotide,” “oligo,” “oligonucleic acid,” “nucleic acid molecule,” and “polynucleotide” may refer to a polymer molecule of nucleoside monomers. Such oligonucleotides may be connected via phosphate linkages or sulfur-containing linkages, and may include deoxyribonucleic acids (DNA), ribonucleic acids (RNA), non-canonical nucleic acids or bases, unnatural or synthetic nucleic acids, or other nucleosides, as well as sugars or other moieties. Such oligonucleotides may further include terminators configured to prevent extension reactions, and which may be removed before addition of subsequent nucleotides to a growing oligonucleotide chain.

As used herein, a “Digital to Analog Converter group,” “DAC group,” or “DAC” may refer to an electronic device or component capable of converting a digital signal into an analog signal. An “Analog to Digital Converter group,” “ADC group,” or “ADC” may be a device or component capable of converting an analog signal into a digital signal.

As used herein, “pitch size” may refer to a distance between adjacent reaction sites (e.g., microelectrodes, reaction wells, microwells) on an electrochemical chip, as described herein. For example, as described herein, the electrochemical chip may include one or more parallel rows of reaction sites. Pitch size may refer to a distance between adjacent parallel rows and/or a distance between reaction sites within the same row. Some groups of reaction sites that are shown in the figures in a horizontal configuration may be referred to as rows, and some groups of reaction sites that are shown in the figures in a vertical configuration may be referred to as columns. However, the operation of the digital chips and flow cells described herein are independent of the orientation of the chips and flow cells. Accordingly, a group of reaction sites may be referred to as either columns or rows, regardless of the orientation of the group to a gravitational plane (e.g., a plane normal to gravitational vectors).

As used herein, “flow cell” may refer to a slide, platform, or other structure including one or more channels configured to receive and pass fluids. The flow cell may include one or more surfaces that are coupled to one or more oligonucleotides, and may be used in the production and/or sequencing of synthetic oligonucleotides.

### Electrochemical Chips

Electrochemical synthesis of oligomers, peptides, and other biomolecules may occur via addition of building blocks or monomers in a cyclic manner, with multiple chemical steps in between. A monomer on a growing chain may be deblocked before a subsequent monomer can be added on. This deblocking process may include a chemical reaction, the reactants for which may be generated via an electrochemical reaction. An exemplary electrochemical reaction may include electrodes (e.g., an anode and a cathode) configured to induce the electrochemical reaction. A potential difference may be applied to the electrodes (e.g., for a given duration), to initiate an electrochemical reaction. The electrochemical reaction results in electron transfer from an oxidation reagent to a first electrode (e.g., anode), and electron transfer from a second electrode (e.g., cathode) to a reduction reagent. An electrochemical system may be designed so that electron transfer to the reduction reagent forms the oxidation reagent.

In some embodiments, quinones, such as hydroquinone and benzoquinone, are used for electrochemical deblocking of previous monomers on the growing strand or chain of an oligomer. Hydroquinone undergoes a 2-electron oxidation to produce benzoquinone and protons at the anode, and the reverse reaction takes place at the cathode to quench the protons generated. The protons may then be used for the deblocking reaction on the existing chain to allow for the coupling of the new monomer onto the growing chain. Upon application of a voltage, these quinones are known to passivate the surfaces of platinum and other electrodes, thereby reducing the efficiency of transfer of electrons to molecules in solution. The passivating layer forms a parallel resistive circuit in terms of an electrical circuit analogy and reduces the total current (charge) transferred for the specific electrochemical reaction of interest.

The present disclosure describes a method for quantifying this passivation using a secondary probe, and developing a feedback mechanism to increase the applied voltage to provide the requisite conditions (e.g., charge) for a specific electrochemical reaction to perform deblocking.

The present disclosure includes one or more electrochemical chips. Electrochemical chips may be used in flow cell assemblies and systems. For example, the electrochemical chips may be used as substrates for oligonucleotide synthesis and/or multipurpose sensors.

In some embodiments, the electrochemical chip may include a silicon chip. In some embodiments, the electrochemical chip may include one or more surfaces functionalized for association with a building block of an oligonucleotide molecule. The building block may include a nucleotide, a dinucleotide, a trinucleotide, and/or another polynucleotide. In addition or alternatively, the building block may include a sugar, a nitrogenous base, a phosphate, or a combination thereof. In some embodiments, the building block may include a nitrogenous base protected with a suitable protecting group and/or blocking group. In addition or alternatively, the building block may include a sugar protected using a suitable protecting group and/or blocking group.

The electrochemical chip provided herein may include a plurality of reaction sites, each of which may be the site of an electrochemical reaction. The reaction sites may be arranged within a primary area of the electrochemical chip.

An electrochemical reaction, such as that occurring on an electrochemical chip, may be facilitated by an electrochemically reactive solution. The electrochemically reactive solution may include an aqueous solution. The electrochemically reactive solution may include one or more reduction reactants, one or more oxidation reactants, one or more bases, one or more electrolytes, one or more diluents, or a combination thereof. The electrochemically reactive solution may include the primary deblocking solution.

A reduction reactant of the electrochemically reactive solution may include a reactant capable of receiving one or more electrons, such as, for example, receiving one or more electrons from a cathode. The electrochemically reactive solution may include greater than or equal to approximately 1 mM, and/or less than or equal to approximately 25 mM, of the one or more reduction reactants. For example, the electrochemically reactive solution may include approximately 1 mM to approximately 15 mM, approximately 5 mM to approximately 25 mM, approximately 5 mM to approximately 15 mM, or approximately 10 mM of the one or more reduction reactants. The one or more reduction reactants may include benzoquinone, quinone derivatives, p-benzoquinone, 2,5-di-tert-butylbenzoquinone, tetrachloro-1,4-benzoquinone, tetrafluoro-1,4-benzoquinone, 2,5-dichloro-benzoquinone, 2,3-dimethoxy-5-methyl-p-benzoquinone, 2,6-diaminoanthraquinone, hexaketocyclohexane, 2,3-dichloro-5,6-dicyano-p-benzoquinone, ferricyanide, ferrocenium ion, or ferrocenemethanol.

An oxidation reactant of the electrochemically reactive solution may include a reactant capable of donating one or more electrons, such as, for example, donating one or more electrons to an anode. The electrochemically reactive solution may include greater than or equal to 250 mM, and/or less than or equal to 1.0 M, of the one or more oxidation reactants. For example, the electrochemically reactive solution may include approximately 250 mM to approximately 1.0 M, approximately 250 mM to approximately 750 mM, approximately 250 mM to approximately 500 mM, approximately 500 mM to approximately 750 mM, or approximately 500 mM of the one or more oxidation reactants. The one or more oxidation reactants may include hydroquinone, 2,5-di-tert-butylhydroquinone, tetrachloro-hydroquinone, tetrafluoro-hydroquinone, hexahydroxy benzene, ferrocyanide, or ferrocene.

In some embodiments, the one or more reduction reactants and one or more oxidation reactants of the electrochemically reactive solution may be paired redox reactants. For example, a reduction reactant (e.g., p-benzoquinone), after accepting one or more electrons, may become the oxidation reactant (e.g., hydroquinone). Similarly, the oxidation reactant (e.g., hydroquinone), after donating one or more electrons, may become the reduction reactant (e.g., p-benzoquinone). The electrochemically reactive solution may include a concentration of oxidation reactants that is greater than a concentration of reduction reactants in the electrochemically reactive solution. For example, the concentration of oxidation reactants in the electrochemically reactive solution may be at least 10 times, at least 20 times, or at least 50 times, inclusive the concentration of the reduction reactants in the electrochemically reactive solution. In some embodiments, methods of the present disclosure may be successfully performed with ionic species, and not paired reduction and oxidation reactants.

The one or more bases of the electrochemically reactive solution may reduce, inhibit, and/or prevent the occurrence of acid-quenching reactions near an electrode (e.g., an anode). The electrochemically reactive solution may include greater than or equal to approximately 0.1 mM, and/or less than or equal to approximately 10 mM, of the one or more bases. For example, the electrochemically reactive solution may include about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 5 mM, or about 10 mM, inclusive of the one or more bases. The one or more bases may include 2,6-lutidine, N,N-diisopropylethylamine (DIPEA), 1,8-diazabicyclo[5.4.0]undec-7-ene (DBU), or pyridine.

The one or more electrolytes of the electrochemically reactive solution may facilitate ion transport between electrodes (e.g., ion transport between a cathode and an anode). The electrochemically reactive solution may include greater than or equal to 5 mM, and/or less than or equal to 50 mM, of the one or more electrolytes. For example, the electrochemically reactive solution may include about 5 mM to about 25 mM, about 10 mM to about 25 mM, about 10 to about 20 mM, about 15 mM to about 25 mM, or about 15 mM to about 20 mM, of the one or more electrolytes. The one or more electrolytes may be non-corrosive with regard to electrode material. The one or more electrolytes may include tetrabutylammonium hexafluorophosphate (TBAPF₆), tetraalkylammonium hexafluorophosphate, tetraalkylammonium paratoluene sulfonate, tetrafluoroborate, tetraphenylborate, perchlorate, tetrachloroferrate, hexafluoroarsenate, potassium chloride, silver chloride, or sulfate.

The electrochemically reactive solution may include one or more diluents. The one or more diluents may constitute the bulk of the electrochemically reactive solution. A diluent may be selected such that the other components of the electrochemically reactive solution are soluble in the electrochemically reactive solution. The one or more diluents may be a liquid, such as, for example, a liquid that is not electrically conductive. The one or more diluents may be non-corrosive, or otherwise inert to electrode materials at the reaction site. The one or more diluents may include water, acetonitrile, methanol, ethanol, dichloromethane, chloroform, dimethylformamide, ethylene glycol, or propylene carbonate.

In some embodiments, the electrochemically reactive solution may include about 10 mM p-benzoquinone, about 500 mM hydroquinone, about 0.5 mM 2,6-lutidine, about 17 mM TBAPF₆, in an acetonitrile solution.

The electrochemically reactive solution described herein may allow for completion of a current-carrying circuit at a reaction site on an electrochemical chip. In some embodiments, a reaction site of an electrochemical chip may include a microwell including two electrodes. The two electrodes may include an anode and a cathode. In addition or alternatively, the microwell may include other electrode architectures, e.g., planar 2D electrodes. An electrochemical reaction may occur at the reaction site (e.g., in a microwell). For example, an electrochemical reaction may occur as a result of application of a voltage pulse to electrodes of the reactive site. The electrochemical reaction may result in the transfer of one or more electrons from a cathode to one or more components of the electrochemically reactive solution (e.g., a reduction reactant). In addition or alternatively, the electrochemical reaction may result in the transfer of one or more electrodes from one or more components of the electrochemically reactive solution (e.g., an oxidation reactant) to an anode. The net current of each reaction site or a subset of reaction sites may be measured and/or recorded.

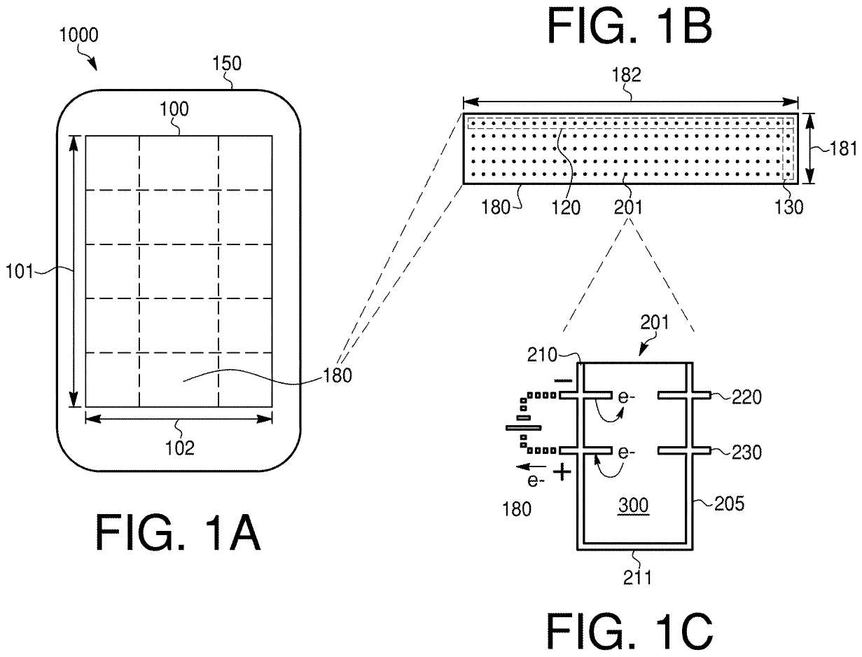

FIG. 1A depicts a partial view of an exemplary electrochemical chip, according to aspects of the present disclosure. Referring to FIG. 1A, an electrochemical chip 1000 may include an active area 100 and an inactive area 150. In some embodiments, the inactive area 150 may partially or fully surround the active area 100. Although FIG. 1A shows the electrochemical chip 1000 with the active area 100 that is rectangular in shape, the active area 100 may be in any shape, such as, for example, a circle, an ellipse, or any other contiguous region or group of contiguous regions. Portions of the inactive area 150 may extend past one or more edges of the active area 100. In some embodiments, the electrochemical chip 1000 may include more than one active area 100, where the active areas 100 are separated by one or more inactive area 150. Furthermore, electrochemical chip 1000 may also include an electrical connection (not shown).

The active area 100 may have a first length 101 and a second length 102 about perpendicular to and shorter than the first length. The first length 101 may be greater than or equal to about 15 mm, and/or less than or equal to about 50 mm. For example, the first length 101 may be about 15 mm to about 16 mm, about 15 mm to about 17 mm, about 15 mm to about 18 mm, about 15 mm to about 19 mm, about 15 mm to about 20 mm, about 15 mm to about 21 mm, about 15 mm to about 22 mm, about 15 mm to about 25 mm, about 15 mm to about 28 mm, about 15 mm to about 30 mm, about 15 mm to about 32 mm, about 15 mm to about 35 mm, about 15 mm to about 38 mm, about 15 mm to about 40 mm, about 18 mm to about 19 mm, about 18 mm to about 20 mm, about 18 mm to about 21 mm, about 18 mm to about 22 mm, about 18 mm to about 25 mm, about 18 mm to about 28 mm, about 18 mm to about 30 mm, about 18 mm to about 32 mm, about 18 mm to about 35 mm, about 18 mm to about 38 mm, about 18 mm to about 40 mm, about 20 mm to about 21 mm, about 20 mm to about 22 mm, about 20 mm to about 25 mm, about 20 mm to about 28 mm, about 20 mm to about 30 mm, about 20 mm to about 32 mm, about 20 mm to about 35 mm, about 20 mm to about 38 mm, about 20 mm to about 40 mm, about 25 mm to about 28 mm, about 25 mm to about 30 mm, about 25 mm to about 32 mm, about 25 mm to about 35 mm, about 25 mm to about 38 mm, about 25 mm to about 40 mm, about 30 mm to about 32 mm, about 30 mm to about 35 mm, about 30 mm to about 40 mm, about 35 mm to about 40 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 25 mm, about 28 mm, about 30 mm, about 32 mm, about 35 mm, about 38 mm, or about 40 mm.

Additionally, the first length may be greater than or equal to about 15 mm, greater than or equal to about 15.5 mm, greater than or equal to about 16 mm, greater than or equal to about 16.5 mm, greater than or equal to about 17 mm, greater than or equal to about 17.5 mm, greater than or equal to about 18 mm, greater than or equal to about 18.5 mm, greater than or equal to about 19 mm, greater than or equal to about 19.5 mm, greater than or equal to about 20 mm, greater than or equal to about 20.5 mm, greater than or equal to about 21 mm, greater than or equal to about 21.5 mm, greater than or equal to about 22 mm, greater than or equal to about 25 mm, greater than or equal to about 28 mm, greater than or equal to about 30 mm, greater than or equal to about 32 mm, greater than or equal to about 35 mm, greater than or equal to about 38 mm, or greater than or equal to about 40 mm.

Furthermore, the first length may be less than or equal to about 15.5 mm, less than or equal to about 16 mm, less than or equal to about 16.5 mm, less than or equal to about 17 mm, less than or equal to about 17.5 mm, less than or equal to about 18 mm, less than or equal to about 18.5 mm, less than or equal to about 19 mm, less than or equal to about 19.5 mm, less than or equal to about 20 mm, less than or equal to about 20.5 mm, less than or equal to about 21 mm, less than or equal to about 21.5 mm, less than or equal to about 22 mm, less than or equal to about 25 mm, less than or equal to about 28 mm, less than or equal to about 30 mm, less than or equal to about 32 mm, less than or equal to about 35 mm, less than or equal to about 38 mm, or less than or equal to about 40 mm.

The second length 102 may be greater than or equal to about 15 mm, and/or less than or equal to about 50 mm. For example, the second length 102 may be about 15 mm to about 16 mm, about 15 mm to about 17 mm, about 15 mm to about 18 mm, about 15 mm to about 19 mm, about 15 mm to about 20 mm, about 15 mm to about 21 mm, about 15 mm to about 22 mm, about 15 mm to about 25 mm, about 15 mm to about 28 mm, about 15 mm to about 30 mm, about 15 mm to about 32 mm, about 15 mm to about 35 mm, about 15 mm to about 38 mm, about 15 mm to about 40 mm, about 18 mm to about 19 mm, about 18 mm to about 20 mm, about 18 mm to about 21 mm, about 18 mm to about 22 mm, about 18 mm to about 25 mm, about 18 mm to about 28 mm, about 18 mm to about 30 mm, about 18 mm to about 32 mm, about 18 mm to about 35 mm, about 18 mm to about 38 mm, about 18 mm to about 40 mm, about 20 mm to about 21 mm, about 20 mm to about 22 mm, about 20 mm to about 25 mm, about 20 mm to about 28 mm, about 20 mm to about 30 mm, about 20 mm to about 32 mm, about 20 mm to about 35 mm, about 20 mm to about 38 mm, about 20 mm to about 40 mm, about 25 mm to about 28 mm, about 25 mm to about 30 mm, about 25 mm to about 32 mm, about 25 mm to about 35 mm, about 25 mm to about 38 mm, about 25 mm to about 40 mm, about 30 mm to about 32 mm, about 30 mm to about 35 mm, about 30 mm to about 40 mm, about 35 mm to about 40 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 25 mm, about 28 mm, about 30 mm, about 32 mm, about 35 mm, about 38 mm, about 40 mm, or greater than or equal to about 15 mm.

Additionally, the second length 102 may be greater than or equal to about 15.5 mm, greater than or equal to about 16 mm, greater than or equal to about 16.5 mm, greater than or equal to about 17 mm, greater than or equal to about 17.5 mm, greater than or equal to about 18 mm, greater than or equal to about 18.5 mm, greater than or equal to about 19 mm, greater than or equal to about 19.5 mm, greater than or equal to about 20 mm, greater than or equal to about 20.5 mm, greater than or equal to about 21 mm, greater than or equal to about 21.5 mm, greater than or equal to about 22 mm, greater than or equal to about 25 mm, greater than or equal to about 28 mm, greater than or equal to about 30 mm, greater than or equal to about 32 mm, greater than or equal to about 35 mm, greater than or equal to about 38 mm, greater than or equal to about 40 mm, or less than or equal to about 15.5 mm.

Furthermore, the second length may also be less than or equal to about 16 mm, less than or equal to about 16.5 mm, less than or equal to about 17 mm, less than or equal to about 17.5 mm, less than or equal to about 18 mm, less than or equal to about 18.5 mm, less than or equal to about 19 mm, less than or equal to about 19.5 mm, less than or equal to about 20 mm, less than or equal to about 20.5 mm, less than or equal to about 21 mm, less than or equal to about 21.5 mm, less than or equal to about 22 mm, less than or equal to about 25 mm, less than or equal to about 28 mm, less than or equal to about 30 mm, less than or equal to about 32 mm, less than or equal to about 35 mm, less than or equal to about 38 mm, or less than or equal to about 40 mm.

The active area 100 may be divided into a plurality of blocks 180, each of the block 180 including a plurality of reaction sites 201. As described in greater detail below and shown in FIG. 1B, each block 180 may include a DAC Group. Each block 180 may have a block length 181 and a block width 182. In some embodiments, the reaction sites 201 may be evenly distributed within the blocks 180, e.g., have substantially equal distances (pitch lengths) between adjacent reaction sites, along block length 181. In another embodiment, the reaction sites 201 may be unevenly distributed along block length 181. In another embodiment, the plurality of reaction sites 201 may be unevenly distributed along block width 182. For example, reaction sites 201 may be arranged in an array of rows 120 and columns 130, wherein each row 120 of reaction sites 201 is evenly spaced along block length 181 and each column 130 of reaction sites 201 is evenly spaced along block width 182. A pitch between adjacent reaction sites 201 in the same row 120 may be greater than or equal to approximately 0.1 μm and/or less than or equal to approximately 3.0 μm. For example, a pitch between adjacent reaction sites 201 in the same row 120 may be approximately 0.1 μm to approximately 2.0 μm, approximately 0.1 μm to approximately 1.5 μm, approximately 0.1 μm to approximately 1.0 μm, approximately 0.5 μm to approximately 2.5 μm, approximately 0.5 μm to approximately 2.0 μm, approximately 0.5 μm to approximately 1.5 μm, approximately 1.0 μm to approximately 2.0 μm, approximately 0.5 μm to approximately 1.0 μm, or approximately 1.0 μm.

Furthermore, a pitch between adjacent reaction sites 201 in the same column 130 may be greater than or equal to approximately 0.1 μm and/or less than or equal to approximately 3.0 μm. For example, a pitch between adjacent reaction sites 201 in the same column 130 may be approximately 0.1 μm to approximately 2.0 μm, approximately 0.1 μm to approximately 1.5 μm, approximately 0.1 μm to approximately 1.0 μm, approximately 0.5 μm to approximately 2.5 μm, approximately 0.5 μm to approximately 2.0 μm, approximately 0.5 μm to approximately 1.5 μm, approximately 1.0 μm to approximately 2.0 μm, approximately 0.5 μm to approximately 1.0 μm, or approximately 1.0 μm.

Additionally, within a block 180, a pitch between adjacent reaction sites 201 in the same row 120 may be greater than, equal to, or less than a pitch between adjacent reaction sites 201 in the same column 130.

As described above, the electrochemical chip 1000 may include a plurality of reaction sites 201 arranged within the active area 100 of the electrochemical chip 1000. FIG. 1C depicts one of the reaction sites 201 in block 180 shown in FIG. 1B, according to aspects of the present disclosure. Each reaction site 201 may include a microwell. As shown in FIG. 1C, the microwell may be configured to retain diluent, and/or an electrochemically reactive solution 300. The microwell may include a bottom surface 211 and an opening 210. Opening 210 may be opposite bottom surface 211. A side wall 205 may extend between the bottom surface 211 and opening 210 of each microwell. Opening 210 may be configured to receive the electrochemically reactive solution 300. The microwell may include two electrodes. For example, the microwell may include electrodes disposed along the side wall 205. The two electrodes may include an anode 230 and a cathode 220. A distance between the anode 230 and the bottom surface 211 may be different than a distance between the cathode 220 and the bottom surface 211. Each electrode may extend around an interior circumference of the microwell or be stamped along the interior wall of the microwell. Although the reaction site 201 shows a cathode 220 above an anode 230, each microwell may be configured such that the anode 230 is above the cathode 220. Each microwell may be configured such that when the internal volume of the microwell is occupied by the electrochemically reactive solution 300, both the cathode 220 and the anode 230 are in contact with electrochemically reactive solution 300.

When both the cathode 220 and the anode 230 are in contact with the electrochemically reactive solution 300 and a voltage is applied across the cathode 220 and the anode 230, a redox reaction may be initiated within the electrochemically reactive solution 300. During the redox reaction, electrons may be transferred from the cathode 220 to the electrochemically reactive solution 300 (e.g., a reduction reactant within the solution) while electrons are transferred from the electrochemically reactive solution 300 to the anode 230 (e.g., an oxidation reactant within the solution). This transfer of electrons may result in the formation of a circuit between the cathode 220 and the anode 230 that provides an observable faradaic current.

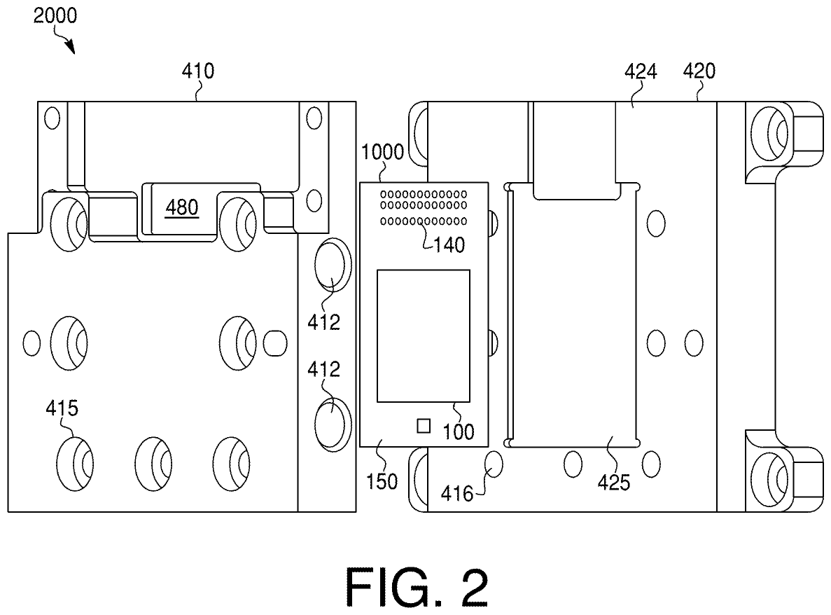

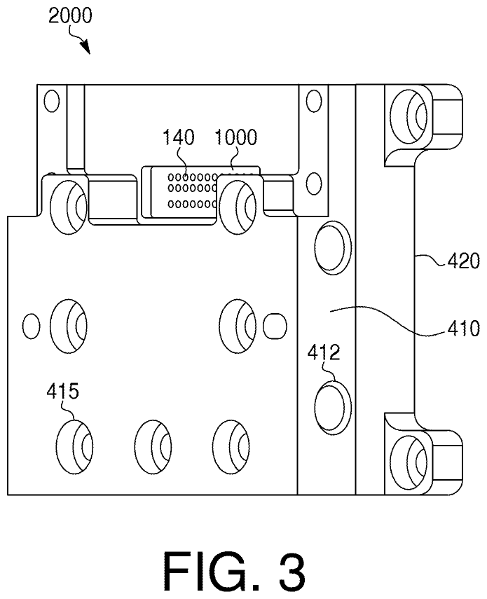

Referring to FIGS. 2 and 3, the electrochemical chip 1000 may be a component of a flow cell assembly 2000. For example, flow cell assembly 2000 may include the electrochemical chip 1000, a flow cell top 410, and a flow cell bottom 420. FIG. 2 shows an exploded view of the flow cell assembly 2000, and FIG. 3 shows an assembled flow cell assembly 2000, where the flow cell top 410 forms a fluidic seal with the flow cell bottom 420. As used herein, the terms “top” and “bottom” are used to distinguish between components of exemplary flow cell assemblies 2000, and do not necessarily imply an orientation of components in the flow cell assembly 2000. Thus, for example, the flow cell assembly 2000 may be assembled such that the flow cell bottom 420 is above the flow cell top 410, the flow cell bottom 420 is parallel to the flow cell top 410, or any other configuration where the flow cell top 410 and the flow cell bottom 420 form a fluidic seal.

The flow cell top 410 may include one or more fluid ports 412. In some embodiments, the flow cell top 410 may include at least four fluid ports 412. In some instances, two of the four fluid ports 412 may be on one side of the flow cell top 410, and the other two may be on the opposite side of the flow cell top 410. The fluid ports 412 may be configured to fluidly couple with a fluid delivery system to facilitate the delivery of the fluids therethrough. The fluid ports 412 may be configured to pass fluid into and out of the flow cell assembly 2000. For example, the fluid ports 412 may be configured to allow fluid to pass from an exterior of the flow cell assembly 2000 to an interior of the flow cell assembly 2000 (e.g., to electrochemical chip 1000 within flow cell assembly 2000). As the fluid passes from the exterior of the flow cell assembly 2000 to the interior of the flow cell assembly 2000, the fluid may fill and/or exit the microwells. In addition or alternatively, the fluid ports 412 may be configured to allow fluid to pass from the interior of flow cell assembly 2000 to the exterior of flow cell assembly 2000. For example, fluid ports 412 may allow for a bidirectional flow of fluid (e.g., diluent and/or electrochemically reactive solution 300) into and from the electrochemical chip 1000 within the flow cell assembly 2000. As another example, the fluid ports 412 may be configured to allow fluid to pass from an exterior of the flow cell assembly 2000 to an interior of the flow cell assembly 2000, and may fill the microwells of the electrochemical chip 1000. Fluid may then flow out of the electrochemical chip 1000, moving from the interior of the flow cell assembly 2000 into the exterior of the flow cell assembly 2000.

The flow cell top 410 may include one or more fastener ports 415. Each fastener port 415 may be configured to receive a fastener (e.g., a screw, a bolt, etc.). The fastener port 415 may allow for the fastener to pass through the flow cell top 410 and into a fastener receiver 416 of the flow cell bottom 420. In some embodiments, the fastener ports 415 and the fastener receivers 416 of the flow cell assembly 2000 are configured such that the electrochemical chip 1000 is surrounded on all sides by fasteners. The fastener port 415 is further described below.

The electrochemical chip 1000 may include an electrical connection 140. The electrical connection 140 may facilitate the transfer of electric signal from a current source (e.g., a current source external to electrochemical chip 1000) to the one or more electrodes (e.g., cathodes 220 and/or anodes 230) of the electrochemical chip 1000. The electrical connection 140 may allow for a computing device to connect with, transmit to, and/or receive signals from the electrochemical chip 1000. The flow cell top 410 may include a connection aperture 480 configured to allow access to the electrical connection 140 from the exterior of the flow cell assembly 2000. For example, referring to FIG. 3, when the flow cell assembly 2000 is in an assembled state such that a fluidic seal is formed between the flow cell top 410 and the flow cell bottom 420, the electrical connection 140 is accessible through the connection aperture 480.

Referring again to FIG. 2, the flow cell bottom 420 may include a bottom interface surface 424. When the flow cell assembly 2000 is assembled and a fluidic seal is formed between the flow cell top 410 and the flow cell bottom 420, the bottom interface surface 424 may be in contact with the flow cell top 410. The flow cell bottom 420 may include a recess 425. The recess 425 may be configured to receive the electrochemical chip 1000. For example, the recess 425 may be approximately the same size and dimensions, or alternatively slightly bigger in one or more dimensions than the dimensions of the electrochemical chip 1000, such that the electrochemical chip 1000 can be positioned between the flow cell top 410 and the flow cell bottom 420 while there is a fluidic seal between the flow cell top 410 and the flow cell bottom 420. In some embodiments, the recess 425 includes a surface that is substantially planar. The recess 425 may be substantially square, rectangular, or circular, such that a shape of the recess 425 substantially matches the shape of the electrochemical chip 1000 received therein.

The flow cell bottom 420 may include one or more fastener receivers 416. The fastener receivers 416 may be configured to receive fasteners that have passed through the fastener ports 415 of the flow cell top 410. For example, the flow cell assembly 2000 may be configured such that the fastener ports 415 of the flow cell top 410 align with fastener receivers 416 of the flow cell bottom 420. As described herein, tightening fasteners within the fastener ports 415 and the fastener receivers 416 may decrease a distance between the flow cell top 410 and the flow cell bottom 420. In addition or alternatively, tightening fasteners within the fastener ports 415 and the fastener receivers 416 may create a fluidic seal between the flow cell top 410 and the flow cell bottom 420.

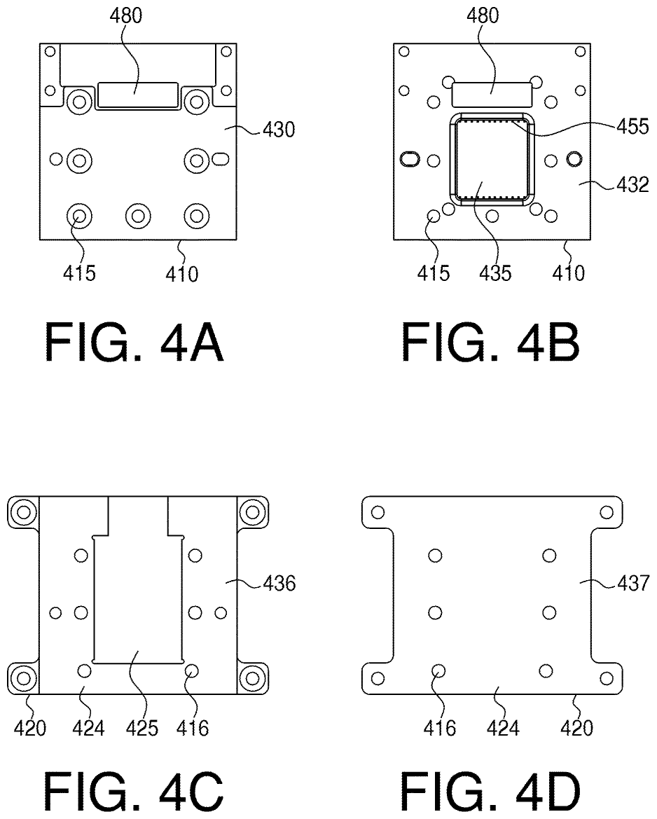

FIG. 4A includes a front view of the flow cell top 410, FIG. 4B includes a rear view of the flow cell top 410, FIG. 4C includes a front view of the flow cell bottom 420, and FIG. 4D includes a rear view of the flow cell bottom 420. Referring to FIGS. 4A and 4B, the flow cell top 410 may include a front surface 430 and a rear surface 432. The fastener ports 415 of the flow cell top 410 may extend from the front surface 430 to the rear surface 432. The flow cell top 410 may include a top recess 435. Distributors 455, described herein, may be disposed in the top recess 435. In addition or alternatively, the distributors 455 may be in fluid communication with the top recess 435. When the flow cell assembly 2000 is assembled, the top recess 435 may align with a portion of the recess 425 of the flow cell bottom 420.

Referring to FIGS. 4C and 4D, the flow cell bottom 420 may include a front surface 436, a rear surface 437, and a bottom interface surface 424. The fastener receivers 416 of the flow cell bottom 420 may extend from the front surface 436 to the rear surface 437. During assembly of the flow cell 2000, the rear surface 432 of the flow cell top 410 may be brought into contact with the front surface 436 of the flow cell bottom 420, such that the fastener receivers 416 are aligned with the fastener ports 415.

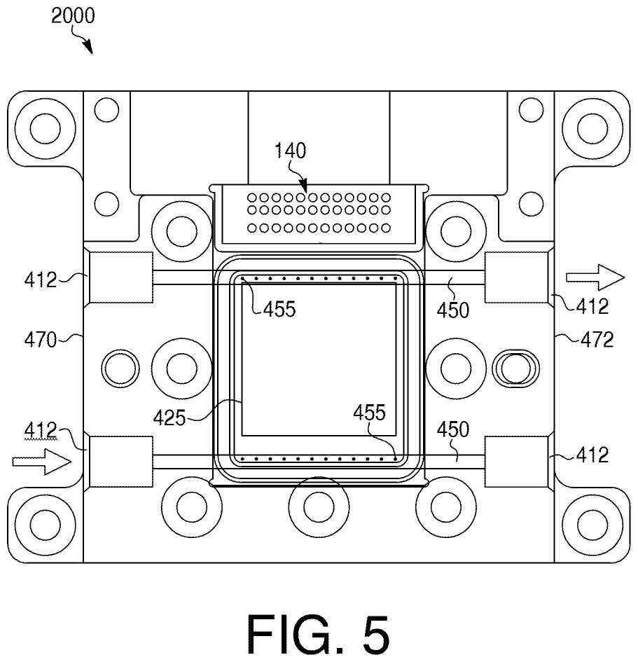

Referring to FIG. 5, each of the one or more fluid ports 412 may be connected to a manifold 450. Each manifold 450 may extend a length from a first fluid port 412a on a first surface 470 of the flow cell assembly 2000 to a second fluid port 412b on a second surface 472 of the flow cell assembly 2000. The manifolds 450 may be entirely within the flow cell top 410. In some embodiments, the manifolds 450 are formed when a fluidic seal is created between the flow cell top 410 and the flow cell bottom 420. The manifold 450 may connect the fluid ports 412 from the first surface 470 of the flow cell assembly 2000 to the second surface 472 of the flow cell assembly 2000, where the second surface 472 is opposite the first surface 470. The manifold 450 may include one or more distributors 455 along a length of the manifold 450. The distributors 455 may allow for fluid to flow from the manifold 450 to the recess 425. In addition or alternatively, the distributors 455 may allow for fluid to flow from the recess 425 to the manifold 450.

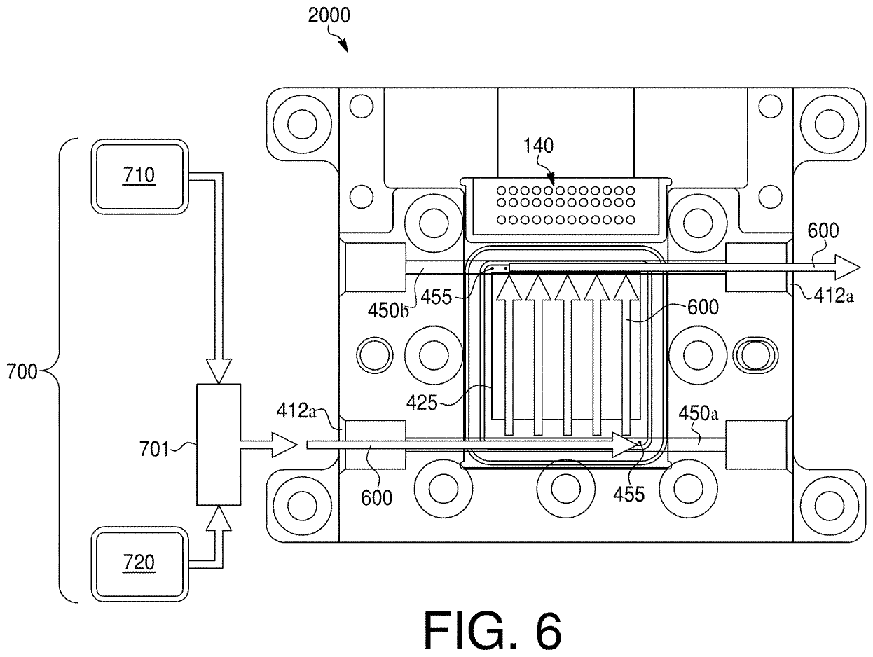

Referring to FIG. 6, an exemplary flow of fluid through the flow cell assembly 2000 is shown by arrows 600. For example, fluid (e.g., diluent or an electrochemically reactive solution 300) may flow from a liquid delivery system 700, through a first fluid port 412a, through a first manifold 450a, and through the distributors 455 to a cavity formed within the recess 425 and/or the top recess 435. Fluid entering the recess 425 and/or the top recess 435 may be distributed along the electrochemical chip 1000 that may be disposed within the cavity formed within the recess 425 and/or the top recess 435 of flow cell assembly 2000. For example, the fluid may enter one or more reaction sites 201 along the electrochemical chip 1000. Still referring to FIG. 6, fluid may flow from the cavity, through the distributor 455 of a second manifold 450b to the second manifold 450b, and from the second manifold 450b through a second fluid port 412 b. Fluid exiting the flow cell assembly 2000 through the fluid port 412 may be passed to another system (e.g., another flow cell assembly 2000), collected, and/or disposed of as waste.

The liquid delivery system 700 may include a pressure-driven delivery mechanism 710 and/or a pump-driven delivery mechanism 720. The pump-driven delivery mechanism 720 may include a syringe pump. The liquid delivery system 700 may include a valve 701 (e.g., a manual valve). The pressure-driven delivery mechanism 710 and the pump-driven delivery mechanism 720 may both be fluidically connected to the valve 701. In addition or alternatively, the valve 701 may be fluidically connected to a fluid port 412. The valve 701 may be actuated to allow fluid from the pressure-driven delivery mechanism 710 to enter the fluid port 412. In addition or alternatively, the valve 701 may be actuated to allow fluid from the pump-driven delivery mechanism 720 to enter the fluid port 412. The valve 701 may be configured such that fluid from only one of the pressure-driven deliver mechanism 710 and the pump-driven delivery mechanism 720 to enter the fluid port 412 at one time. The fluid delivery system 700 may include multiple reservoirs of different fluids (e.g., electrochemically reactive solution 300, diluent, wash reagent, etc.). In some embodiments, the pressure-driven delivery mechanism 710 is connected to a reservoir of one fluid (e.g., diluent), and the pump-driven delivery mechanism 720 is connected to a reservoir of a different fluid (e.g., electrochemically reactive solution 300).

In some embodiments, the liquid delivery system 700 may deliver liquid to the flow cell cavity in fill volume increments. Fill volume increment may be about 0.01 μL to about 40 μL, such as about 0.01 μL, about 0.1 μL, about 0.2 μL, about 0.5 L, about 1 μL, about 2 μL, about 5 μL, about 10 μL, about 20 μL, about 30 μL, or about 40 μL. In some embodiments, the fill volume increment is about 0.5 μL. In some embodiments, the fill volume increment is about 1 μL. In some embodiments, the fill volume increment is about 10 μL. In some embodiments, the fill volume increment is about 20 μL.

In some embodiments, the flow cell assembly 2000 may be placed in a vertical or tilted orientation (e.g., an orientation at an angle relative to the horizon) using a stand or base. The flow cell assembly 2000 may include or may be connectable to a station or platform to which, in some instances, one or more fluid dispensing stations (e.g., a liquid delivery system 700) can be stably associated (e.g., by mounting). In some embodiments, a mount is used to place the flow cell on a stand or base, such that the flow cell assembly can be oriented at one or more angles.

### Systems of Flow Cell Assemblies Comprising Electrochemical Chips

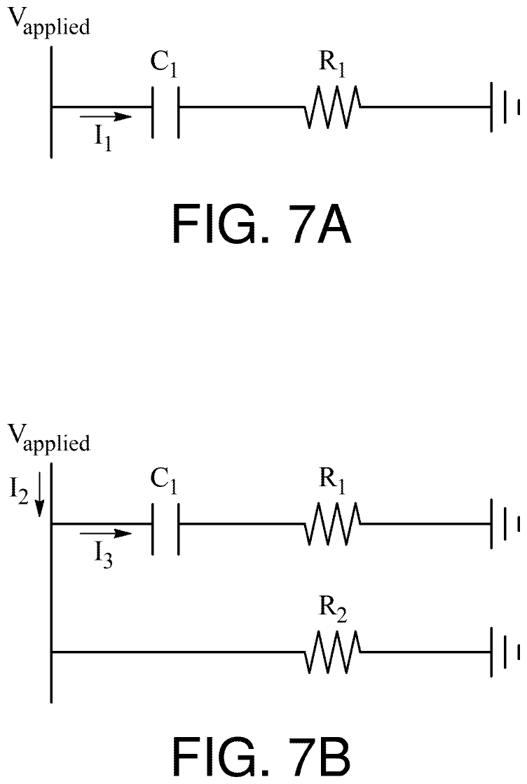

In an electrochemical synthesis reaction, the reagents used for the deblocking of previous monomers on the growing strand of an oligomer, may, upon application of a voltage, passivate the surfaces of platinum and other electrodes thereby reducing the efficiency of transfer of electrons to molecules in solution. The passivating layer forms a parallel resistive circuit in terms of an electrical circuit analogy and reduces the total current (charge) transferred for the specific electrochemical reaction of interest. FIG. 7A illustrates an electrical circuit for charge transfer from electrode (left) to solution (right). C₁ is a double layer capacitance at the electrode-electrolyte interface, and R₁ is a charge transfer resistance to the molecule in solution. This is known as Randle's circuit for electrochemical cells. In contrast, FIG. 7B illustrates a similar electrical circuit with a parallel passivation resistor that causes the total current I₁ to reduce by (I₁−V_{applied}/R₂), where I₁ is the total current without passivation, V_{applied} is the voltage applied, and R₂ is the resistance offered by the passivation layer.

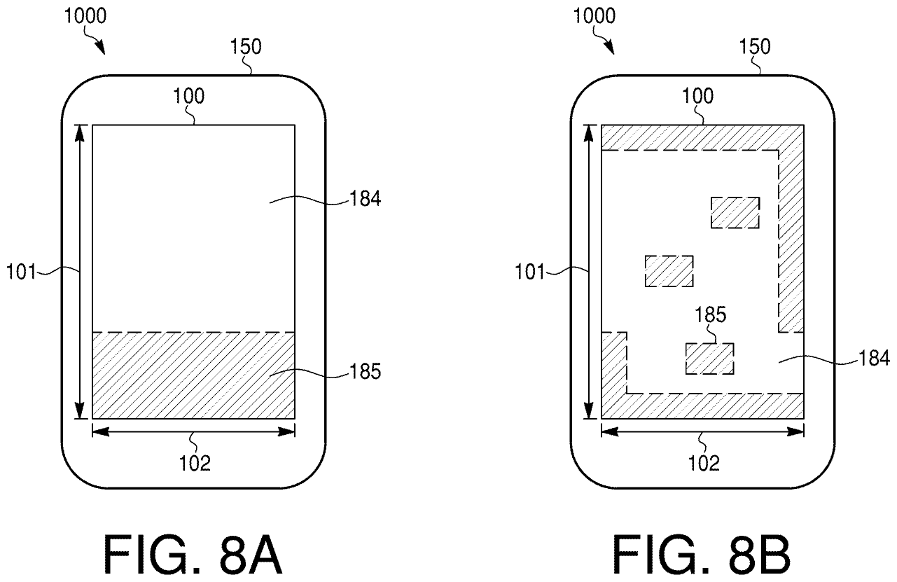

FIG. 8A depicts the electrochemical chip 1000 with the active area 100, and the inactive area 150. The active area 100 may include a primary array 184 of reaction sites, and a secondary array 185 of reaction sites. The primary array 184 may be used to synthesize an end-product. The primary array 184 may be used for performing electrochemical deblocking. Because secondary array 185 is located on the electrochemical chip 1000 and in the same flow cell as primary array 184, both the primary array 184 and the secondary array 185 are exposed to the same chemical reagents and substrates. The secondary array 185 may be used for monitoring the quality of the electrochemical reaction on the primary array 184. In some embodiments, the secondary array 185 may be used for monitoring the quality of the electrochemical reaction on active area 100 via synthesis. In other embodiments, the secondary array 185 may be used for monitoring the quality of the electrochemical reaction in the active area 100 by using electroanalytical techniques without synthesis. A voltage may be applied between two electrodes (e.g., an anode and cathode) to develop a potential difference for performing an electrochemical reaction.

In some embodiments, the secondary array 185 may be probed via a potentiostat for monitoring electrochemical processes and performing electroanalytical techniques. In some instances, the electroanalytical techniques may include cyclic voltammetry. In other instances, the electrochemical techniques may include chronoamperometry. In further instances, the electroanalytical techniques include chronocoulometry. Electrochemical impedance spectroscopy may also be used as an electroanalytical technique.

In some embodiment, the secondary array 185 may have the same architecture as the primary array 184. In other embodiments, the secondary array 185 may have a different architecture than the primary array 184. In further embodiments, the secondary array 185 may also be used for oligonucleotide and other end product synthesis.

FIG. 8B shows other arrangements of the secondary array 185 on the active area 100. In some instance, the secondary array 185 may be located on a top edge of the active area 100, In other embodiments, the secondary array 185 may be located on a bottom edge of the active area 100. Another embodiment may have the secondary array 185 on a left edge of the active area 100. A different embodiment may have the secondary array 185 on a right edge of the active area 100. In other embodiments, more than one secondary array 185 may be placed on the active area 100 to measure the spatial variation in electrochemical performance on the active area 100. The secondary array 185 may occupy about 0.1-99.9% of the area of the active area 100. In some instances, the secondary array 185 may occupy about 0.1-1.0%, about 1.0-10.0%, about 10.0-20.0%, about 20.0-30.0%, about 30.0-40.0%, about 40.0-50.0%, about 50.0-60.0%, about 60.0-70.0%, about 70.0-80.0%, about 80.0-90.0%, or about 90.0-99.9% of active area 100.

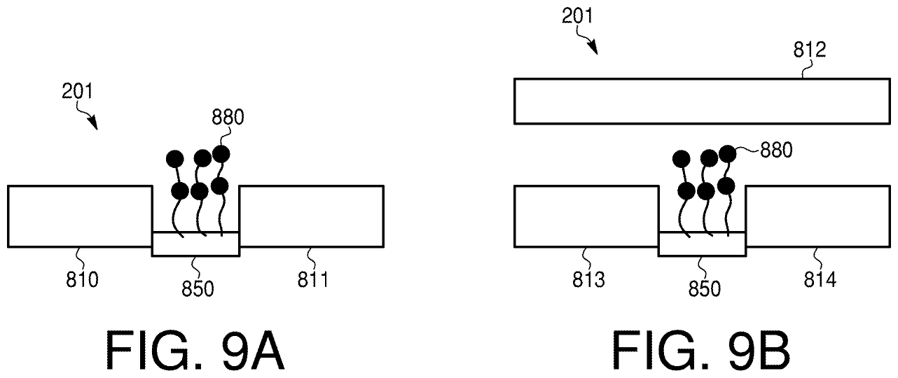

FIG. 9A depicts the reaction site 201 on the primary array 184 with two electrodes 810 and 811. The reaction site 201 may include a microwell 850 disposed between the two electrodes 810 and 811. In some embodiments, both electrodes 810 and 811 may be working electrodes, for example, working electrode 810 may be a cathode, and working electrode 811 may be an anode. In other embodiments, the reaction site 201 may contain 3-, 4-, or more electrode configuration. A potential difference may be applied between two working electrodes, to develop a potential difference and perform an electrochemical reaction to synthesize oligonucleotide 880.

FIG. 9B depicts the reaction site 201 on the secondary array 185. This secondary array 185 may or may not have the same architecture as the primary array 184. The secondary array 185 may also be used for synthesis. FIG. 9B describes an embodiment of an exemplary 3-electrode configuration with the working electrode 813 being probed, a reference electrode 814 close to the working electrode 813 and a counter electrode 812. In some embodiments, the microwell 850 can be disposed between the working electrode 813 and the reference electrode 814. In some instances, the working electrode 813 on the secondary array 185 may be probed by a potentiostat. Other electrode configuration are also possible. In other embodiments, the reaction site 201 may include 3-, 4-, or more electrode configuration.

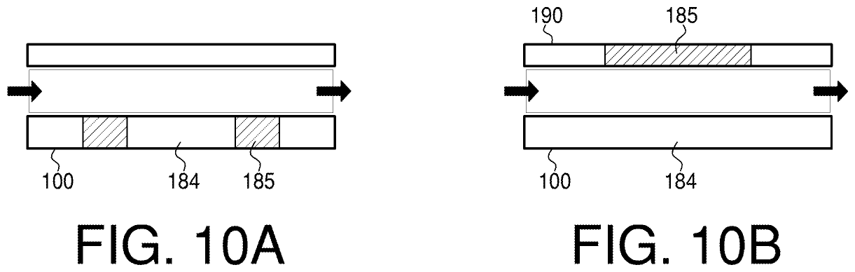

FIGS. 10A and 10B depict different variations of the secondary array 185. In some embodiments, the secondary array 185 may be a part of the active area 100, as shown in FIG. 10A, but controlled separately from the primary array 184. In another embodiment as shown in FIG. 10B, the secondary array 185 may be placed on a flow cell lid 190 of the electrochemical chip 1000, such that the secondary array 185 is a separate entity altogether from the primary array 184, while the secondary array 185 is exposed to substantially the same chemical reagents but is not part of the same substrate as the primary array 184.

The secondary array 185 may have different electrode architectures. Secondary array 185 may include 2D, 2.5D, and 3D electrode architectures. In some embodiments, the secondary array 185 may have different number of electrodes. This could range from one or more electrodes and a combination of several variations within the same architecture. In another embodiment, the secondary array 185 may have different cathode/anode areas in the same secondary array 185. Additionally, the secondary array 185 may have different areas of each working electrode if multiple working electrodes exist in the design.

Various suitable metals may be used in the electrodes of the secondary array 185. These metals may include, but are not limited to gold, platinum, palladium, titanium, indium, copper, and/or nickel. Additionally, semiconductors such as silicon, gallium, and germanium may also be used. In addition or alternatively, any electrochemically active materials that have the capability to donate and accept electrons from redox molecules in solution may be used. The secondary array 185 may also have different permutation and combinations of all the above variations.

A secondary inert redox probe solution may be used to probe the electrochemistry on the secondary array 185 without specifically generating reactants for the electrochemical deblocking reaction. This probe may be used to understand the electrode condition and the effectiveness of its charge transfer to/from the molecule. In some instances, the secondary inert redox probe is ferrocene. Any other redox probe molecule may be used for the purposes of the methods described in this disclosure that do not participate in the deblocking reaction specific to the product being synthesized.

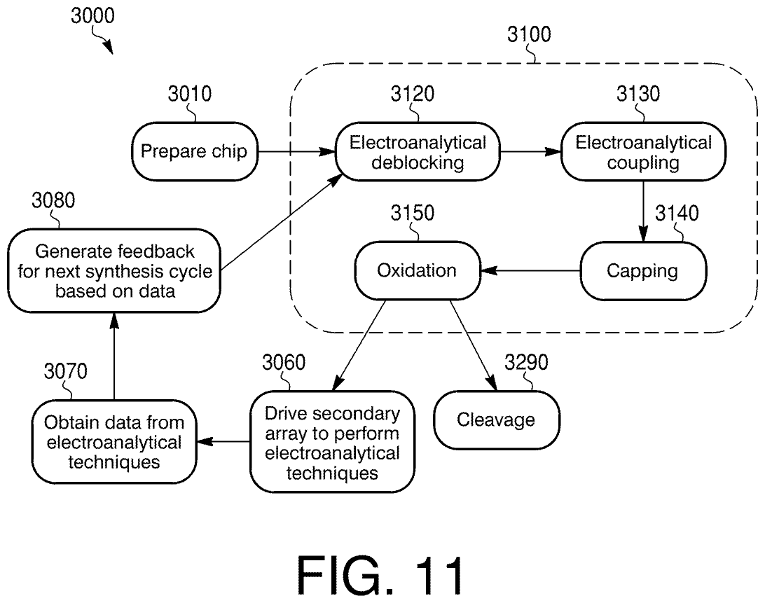

FIG. 11 describes an exemplary workflow 3000 of the methods described in the current disclosure. For example, the workflow 3000 illustrates a method for determining conditions on an electrochemical chip according to some aspects of the current disclosure. At 3010, an electrochemical chip 1000 may be prepared according to the methods described in the current disclosure. Preparation of the electrochemical chip 1000 may include functionalizing its surface for association with a building block of an oligonucleotide molecule. The oligonucleotide molecule may be a blocked oligonucleotide molecule. The electrochemical chip 1000 may include both a primary array 184 and a secondary array 185. At 3100, the electrochemical chip 1000 may undergo a synthesis cycle, which may include steps 3120, 3130, 3140, and 3150. At 3120, electrochemical deblocking may occur. At 3130, an electrochemical coupling may take place, in which a protected nucleoside is coupled with the unblocked oligonucleotide extending from the surface of the electrochemical chip 1000. At 3140, the electrochemical chip 1000 may be capped to block all unreacted OH groups on the surface of the electrochemical chip 1000. At 3150, oxidation may occur. At 3290, the oligonucleotide may be cleaved, and at 3060, the secondary array may be used to perform an electroanalytical technique. At 3070, data may be obtained from the electroanalytical technique, by the computing device. The computing device can compare a feature of the data to a pre-determined threshold and based on the comparison, determine a condition of the electrochemical chip 1000. At 3080, feedback may be generated of the next synthesis cycle, by the computing device, based on the data obtained. The step 3100 may be repeated utilizing the feedback obtained from the earlier cycle. In some embodiments, the computing device may generate a signal indicative of a condition of the electrochemical chip 1000 determined by the computing device. The signal may be communicated to the display information related to the condition, stored in a memory, and/or in a feedback loop to change an operating parameter of the electrochemical chip 1000.

### Electroanalytical Techniques

One or more electroanalytical techniques may be used to assess the conditions of the electrochemical chip. The data received from the electroanalytical technique may be received by a computing device, and compared to a threshold. In some instances, the threshold is a reference curve. In other instances, the threshold is a triangular potential waveform.

### Cyclic Voltammetry (CV)

Cyclic voltammetry (CV) uses a triangular potential waveform to analyze the electrochemical properties of chemical species undergoing oxidation or reduction at an electrode surface. CV involves applying triangular voltage waveforms at a specified scan rate between the working electrodes and reference electrodes for a specified number of cycles. CV measures current as a function of potential. Cyclic voltammetry analysis can provide insight on oxidation and reduction peak potentials (Ep,a and Ep,c) to inform on the energy characteristics of charge transfer. A lower value of oxidation and reduction peak potentials (Ep,a and Ep,c) shows a smaller energy barrier for charge transfer to the redox species compared to a higher value of oxidation and reduction peak potentials (Ep,a and Ep,c) for the same scan rate. Cyclic voltammetry analysis can provide insight on oxidation and reduction peak currents (Ip,a and Ip,c) which are directly proportional to the concentration of the species in solution and can inform on the actual charge transferred to the species as well. Cottrell mass transport curve should give a 1/√t̅ dependence and can inform on any adsorption occurring on the electrode and proper mass transport limited behavior of the system. ΔE is the difference in the oxidation and reduction half-way potentials that gives information on the iR resistance losses, and the number of electrons transferred in the reaction. All the above data inform on the electrochemistry at the electrode-solution interface and any deviation from the ideal signature shown below can be identified and used to gain insight on the chemical phenomena at play.

Other electroanalytical techniques, such as, for example, those described in “Bard, A. J. ; Faulkner, L. R. Electrochemical Methods: Fundamentals and Applications, 2nd ed. ; Wiley: New York, 2001,” which is incorporated by reference herein in its entirety, may also be used to analyze the electrochemical properties of chemical species undergoing oxidation or reduction at an electrode surface. For example, chronoamperometry, chronocoulometry, and/or Electrochemical impedance Spectroscopy (EIS), may be used. In addition or alternatively, galvanostatic measurements techniques such as chronopotentiometry may be used. In additional embodiments, differential pulse voltammetry, square wave voltammetry and spectroelectrochemical methods may be used.

### Applications

### Adjusting Synthesis Parameters

The methods described in this disclosure may be used to monitor electrode quality during end-product synthesis and provide feedback to adjust synthesis parameters. In some embodiments the electroanalytical techniques may be performed on the secondary array 185 to obtain insights on the electrode quality, effectiveness of charge transfer, and electrochemistry at the electrode-electrolyte interface. The results obtained may be received by the computing device and be used as feedback to modify parameters on the electrochemical deblocking step on the primary array 184. The modified parameters may include reduction and oxidation solution concentrations, voltage, and time among other things.

In one embodiment, the primary array 184 may be used to synthesize the end-product, while the secondary array 185 may only be exposed to the secondary inert redox probe. In this embodiment, the secondary array 185 may be exposed to all the chemical reagent steps and fluidic actions as the primary array 184 without an electrochemical deblocking reaction occurring on the secondary array 185. This embodiment may be used for evaluating the impact of the chemical reagents and fluidic actions on the electrode surface/substrate and using the secondary inert redox probe's electroanalytical output as feedback for adjusting synthesis parameters and improving outcomes.

In another embodiment, the primary array 184 and the secondary array 185 may both be used to synthesize the end-product, while the secondary array 185 may also be exposed to the secondary inert redox probe. In this embodiment, the secondary array is exposed to all the chemical reagent steps and fluidic actions as primary array 184 in addition to an electrochemical deblocking reaction occurring on secondary array 185. This embodiment may be used for evaluating the impact of the chemical reagents and fluidic actions on the electrode surface/substrate, as well as the impact of active deblocking on electrode quality.

In a different embodiment, electroanalytical techniques may be performed on the electrochemical deblocking solution using the secondary array 185. In this embodiment, deblocking may occur at specific sites on the secondary array. This embodiment may provide information on the exact electrochemistry of the deblocking solution. The effectiveness of charge transfer for the deblocking solution of interest may be probed using this method and the insights gained may be used as dynamic feedback during synthesis to improve the synthesis parameters on the active array.

### Creation of a Passivation Curve and Adjusting Voltage Based on Feedback

Methods of the present disclosure may include obtaining feedback from an electroanalytical technique and adjusting voltage of an electrochemical synthesis based on the feedback. For example, a method may include obtaining a reference curve for passivation using the secondary array and the secondary inert redox probe. A reference curve may be obtained by applying a pulse voltage to perform electrochemical deblocking on both the primary array 184 and the secondary array 185. This may be followed by performing chronoamperometry of the secondary inert redox probe (e.g., ferrocene) on the secondary array and obtain current versus time (i vs. t) plots for a given applied voltage (V_{applied}). This process may be repeated for “n” number of cycles involved in making the requisite oligomer. Furthermore, the net oxidation current (μA) for ferrocene may be calculated and plotted against the number of cycles to obtain fit type. The Rₚₐₛₛᵢᵥₐₜᵢₒₙ value may be obtained from this curve since Rₚₐₛₛᵢᵥₐₜᵢₒₙ=V_{applied}/slope of the plot.

In some embodiments, an electroanalytical technique such as chronoamperometry or cyclic voltammetry of the secondary inert redox probe (e.g., ferrocene) may be performed on the secondary array 185 to obtain the oxidation current i1. A pulse voltage may be applied to the primary array 184 and the secondary array 185 to perform electrochemical deblocking. An electroanalytical technique such as chronoamperometry or cyclic voltammetry of the secondary inert redox probe (e.g., ferrocene) may be performed on the secondary array to obtain the oxidation current i2. The difference between i1 and i2 may be calculated wherein Δi=i1−i2. Since the entire process scales linearly, the Δi factor obtained herein corrected with the Rₚₐₛₛᵢᵥₐₜᵢₒₙ obtained from the reference curve, yields ΔV, wherein ΔV=Δi*Rₚₐₛₛᵢᵥₐₜᵢₒₙ. As such, potential may be increased by ΔV. This process may be repeated for “n” number of cycles involved in making the oligomer.

### Assessing Electrode Quality Prior to Synthesis

The methods of this disclosure may be used to evaluate the cleanliness of the active area 100 of the electrochemical chip 1000 prior to the commencement of the synthesis reaction. In one embodiment, electroanalytical techniques may be used on the secondary inert redox probe solution on the secondary array before and after cleaning.

This may be used to verify the electrochemical efficiency of the active area 100 before a cyclic synthesis reaction. Furthermore, this may act as a quality control metric for the electrochemical chip 1000 before synthesis.

### Assessing Electrode Cleaning Methods

Feedback from the electroanalytical technique performed on the secondary inert redox probe on the secondary array 185 may be used to compare several electrode cleaning methods and optimize their specific parameters such as incubation times, number of cleaning cycles until optimal electrode cleaning method is determined. Results obtained herein may also inform on the optimal cleaning protocols for each electrochemical chip used.

### Assessing Deblocking Solutions

The secondary array 185 and the methods described herein can also be used as a tool for evaluating different electrochemical deblocking solutions using electroanalytical techniques. Information on optimal redox potentials may be obtained to help develop better deblocking solutions for electrochemical synthesis.

### Assessing Reuse of Electrode Post-Synthesis and Cleavage of Final Products

As described above, CV graphs of the secondary inert redox probe solution on the secondary array 185 give a good indication of the cleanliness and readiness of the array electrodes for charge transfer reactions. The same method can be used for evaluating electrodes/surfaces when reusing electrochemical chips for synthesis after cleaving the previous product and preparation of the surface. The method developed here gives the true electrochemical efficiency of charge transfer at the electrode-electrolyte interface and acts as a quality metric for reusing chips as well.

### EXAMPLES

### Example 1: Cyclic Voltammogram of Secondary Inert Redox Probe

### Experimental Setup

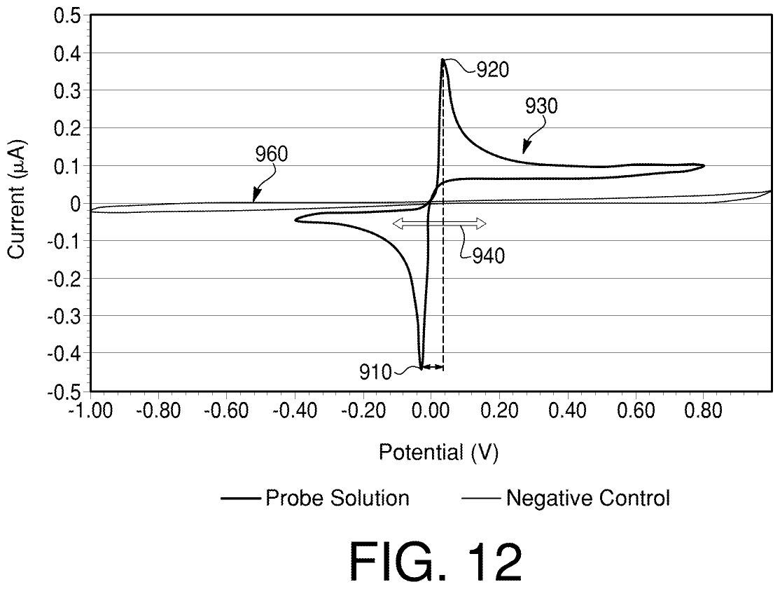

The primary components of the experimental setup include a liquid delivery system, a flow cell containing an exemplary electrochemical chip as described herein, electrical components, and a CPU/PC (e.g., a computing device). The electrochemical chip (or substrate) has the primary and secondary electrode arrays on it. The electrochemical chip is housed in a flow cell and connected to a fluid handler that can cycle reagents through the flow cell. A potentiostat is connected to the electrodes on the secondary array and used for applying the voltage, controlling the potential and measuring current for a standard cyclic voltammetry routine. The computing device may be configured to receive data from the potentiostat, compare a feature of the data to a pre-determined threshold, determine a condition of the electrochemical chip based on the comparison, and generate a signal indicative of the condition. The secondary inert redox probe, including 2.5 uM Ferrocene and 0.1M Tetrabutylammonium hexafluorophosphate dissolved in acetonitrile, was filled onto the substrate containing electrodes, and cyclic voltammetry was performed that generated the CV curve shown in FIG. 12. As a comparison, a negative control without the redox probe, i.e. 0.1M Tetrabutylammonium hexafluorophosphate dissolved in acetonitrile, was also measured the same way.

### Results

FIG. 12 depicts an exemplary cyclic voltammogram (Potential (E) versus current (I) curve) for the secondary inert redox solution on a 3-electrode configuration on the secondary array during synthesis of oligomers on the primary array. The CV curve is compared against a negative control 960 which is composed of the electrolytes used and the diluent. Several insights may be gathered from this curve. For example, the potential (Ep,a) of oxidation peak 920 and the potential of reduction peak 910 (Ep,c) inform on the energy characteristics of charge transfer. A lower value of the potential (Ep,a) of oxidation peak 920 and the potential of reduction peak 910 (Ep,c) shows a smaller energy barrier to charge transfer to the redox species compared to a higher value of the potential (Ep,a) of oxidation peak 920 and the potential of reduction peak 910 (Ep,c) for the same scan rate. The current of oxidation peak 920 (Ip,a) and the current of reduction peak (Ip,c) are directly proportional to the concentration of the species in solution and can inform on the actual charge transferred to the species as well. Cottrell mass transport curve 930 gives a 1/√t̅ dependence and can inform on any adsorption occurring on the electrode and proper mass transport limited behavior of the system. ΔE 940 is the difference in the oxidation and reduction half-way potentials that gives information on the iR resistance losses, and the number of electrons transferred in the reaction. All of the above data inform on the electrochemistry at the electrode-electrolyte interface and any deviation from the ideal signature can be identified and used to gain insight on the chemical phenomena at play.

### Example 2: Cyclic Voltammogram of Primary Deblocking Solution

### Experimental Setup

The setup is similar to Example 1; however, the secondary inert redox probe is replaced with the primary deblocking solution including: 500 mM Hydroquinone, 17 mM Tetrabutylammonium hexafluorophosphate, 10 mM Benzoquinone, and 0.5 mM 2,6-lutidine dissolved in acetonitrile.

### Results

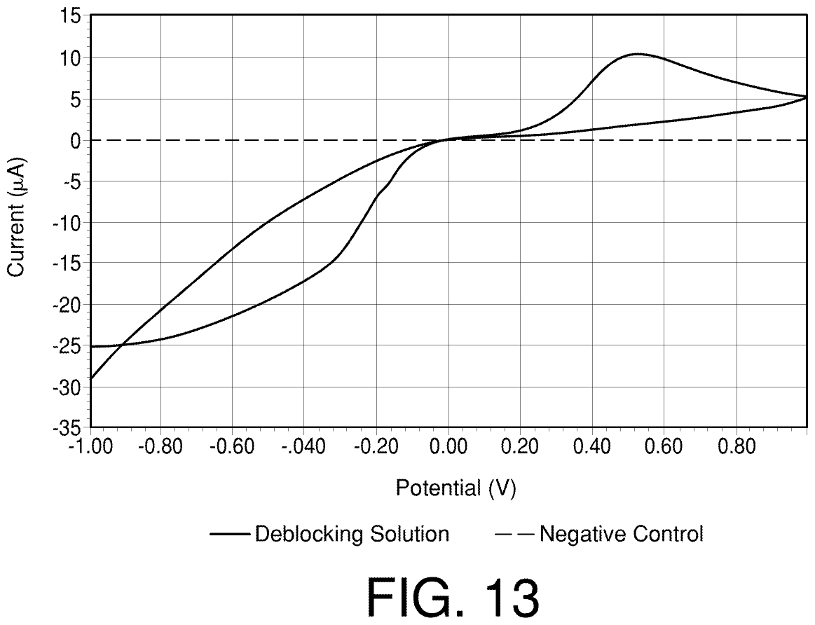

FIG. 13 illustrates the CV curve of the primary deblocking solution in a 3-electrode architecture on the secondary array as described in FIG. 9B. The signature curve is different from the secondary inert redox probe solution as to be expected, due to the use of different solution (secondary inert probe vs. primary deblocking solution), but similar insights and information from example 1 can be gathered from these curves as well.

### Electrode Quality Monitoring and Feedback During Synthesis Using the Secondary Inert Redox Probe

The secondary array may be used to perform electroanalytical techniques on the secondary inert redox probe to obtain insight on the electrode quality, effectiveness of charge transfer and electrochemistry at the electrode-electrolyte interface. These are then used as feedback to modify parameters on the electrochemical deblocking step on the primary array (such as redox concentration, voltages, time, etc.).

### Example 3: Electrode Quality Monitoring During Synthesis on the Primary Array and CV on the Secondary Inert Redox Probe on the Secondary Array

To evaluate the impact of the chemical reagents and fluidic actions on the electrode surface/substrate, the secondary inert redox probe CV output may be used as feedback for improving synthesis.

### Experimental Setup

The experimental setup is similar to Example 1. In this example, the secondary array is exposed to all the chemical reagent steps and fluidic actions as witnessed by the primary array but no synthesis/electrochemical deblocking occurs on the secondary array.

### Results

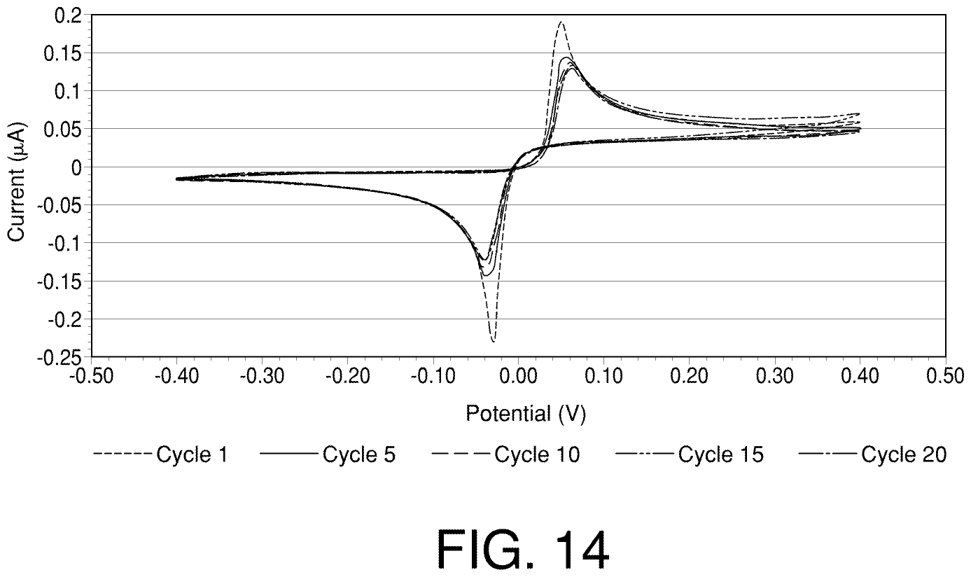

FIG. 14 depicts the electrochemical behavior of secondary inert redox probe CVs on the secondary array during cycles 1, 5, 10, 15, and 20 of oligomer chemical synthesis. The secondary array is subjected to the same fluidic actions as the primary array and the reagents involved in deblocking, capping, oxidation sub steps in synthesis flow over the secondary array as well without specifically electrochemically deblocking on it. As can be observed from FIG. 14, as the number of synthesis cycle increases, the peak currents decrease and the peak oxidation, and peak reduction potentials increase. These observations suggest decreased capability of the electrodes to perform charge transfer to the species in solution, indicating passivation or electrode fouling with synthesis cycling.

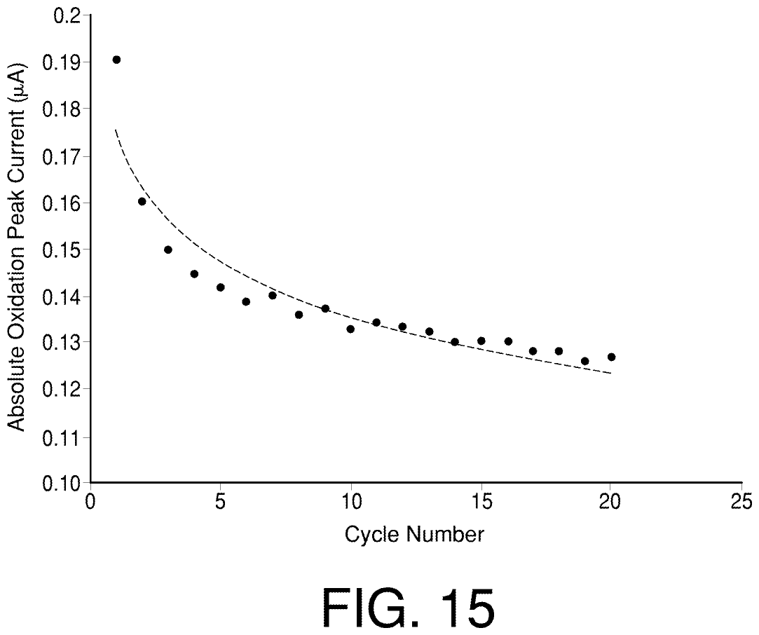

FIG. 15 depicts the absolute oxidation peak current plotted as a function of cycle number from the previous cyclic voltammogram of the secondary redox probe performed during synthesis cycling. As can be observed from the plot of FIG. 15, as the number of cycles increases, the absolute peak current for oxidation decreases. This illustrates a functional relationship of passivation or electrode fouling with cycling as a metric. The function (slope) obtained can be used as feedback to rectify the voltage/concentration or other parameters in the electrochemical deblocking steps for synthesis. This can be used as characterization data for measuring synthesis quality potentially or perform depassivation strategies to improve the electrodes. In order to perform depassivation or mitigate electrode fouling: (a) one could apply higher voltages for the electrochemical deblocking step, (b) perform in-situ cyclic voltammetry based electrode cleaning, (c) perform chemical cleaning of the electrodes, (d) or perform reduction of the oxide layer on the electrodes. This characterization using the secondary array during synthesis provides a view into the synthesis quality, that can help inform ways to make the electrode more efficient in performing electrochemistry.

### Example 4: Electrode Quality Monitoring During Synthesis on Both the Primary Array and Secondary Arrays With CV Performed on the Secondary Inert Redox Probe on the Secondary Array

In this case, the secondary array is also subjected to electrochemical deblocking in addition to all the chemical/fluidic steps of synthesis. The secondary inert redox probe CV can provide insights on the electrode quality due to active deblocking as well in addition to the effect of chemical/fluidic steps.

### Experimental Setup

The experimental setup in this case is similar to Example 1.

### Results

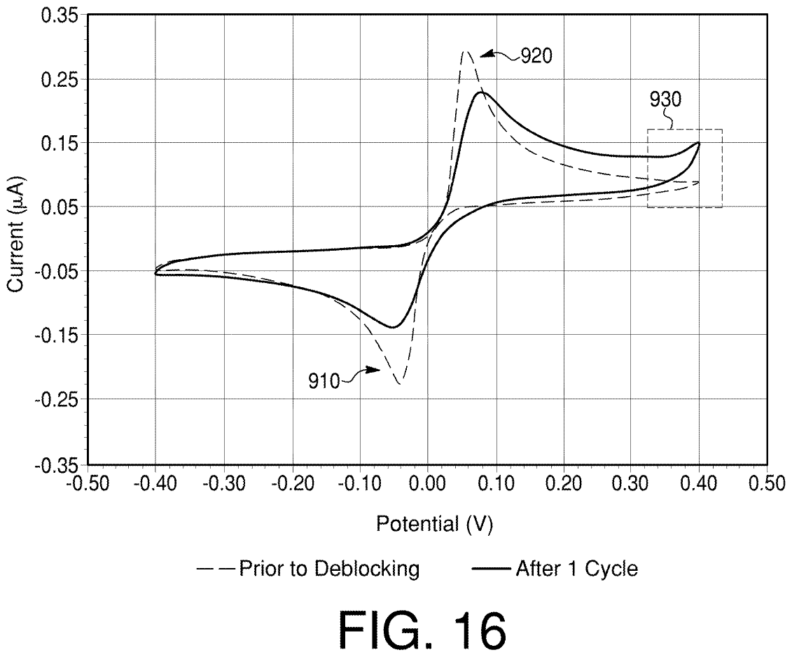

The curve in FIG. 16 depicts the secondary inert redox probe CV before and after the electrochemical deblocking step on the secondary array for 1 cycle. Following 1 cycle of synthesis, changes in the curve can be observed. The increase in oxidation peaks 920 and reduction peak 910 potentials, suggests more energy involved in the charge transfer possibly due to side product generation during electrochemical deblocking such as polymers, nuclei, adsorbed species etc. that can take up the charge in a parallel circuit as well. This increase in potential (ΔV) can be used as an input to the electrochemical deblocking step in the synthesis protocol on the active array since the same effect would be observed on it. Thereby, an increase in voltage on the active array with a translation function may be applied to obtain consistent synthesis quality. The reduction in peak currents for the same CV parameters could imply reduction in the charge transferred for this specific redox probe reaction. This insight could be an input to the concentration of the redox reactants in the electrochemical deblocking step in the active array. As an example, a translation function can be used to predict the increase in concentration to obtain a similar charge transfer magnitude as the previous step. The change in the Cottrell mass transport curve 930 characteristics could imply adsorption-based behavior due to polymer growth/change in substrate characteristics or additional reactions taking place that warrant mitigation or removal with cycling. This insight can be an input to the synthesis process in the primary array to incorporate additional steps that can remove the adsorbed species or improve the substrate surface.

### Example 5: Electrode Quality Monitoring and Feedback During Synthesis Using the Primary Deblocking Solution

In this example, the electroanalytical techniques are performed using the electrochemical deblocking solution on the secondary array with a 3-electrode configuration as shown in FIG. 9B. This would result in deblocking at the specific sites on the secondary array but will provide information on the exact electrochemistry of the deblocking solution. The effectiveness of charge transfer for the deblocking solution of interest can be probed using this method and much like the case above, the insights can be used as dynamic feedback during synthesis to improve on the synthesis parameters on the active array.

### Experimental Setup

The experimental setup in this example was similar to Example 1.

### Results

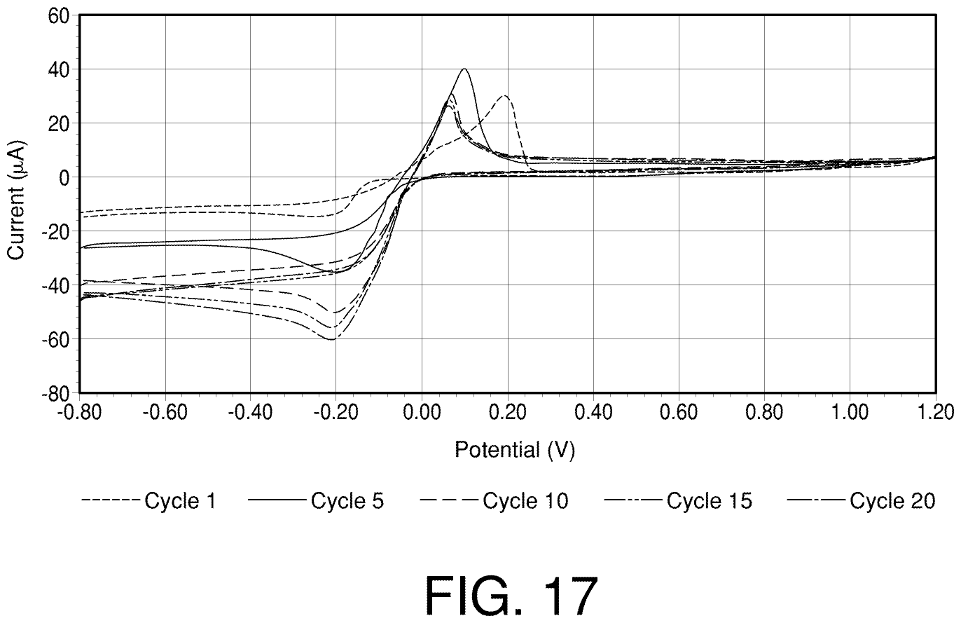

FIG. 17 provides the CV curve for the deblocking solution for 20 synthesis cycles for a 3-electrode configuration of the secondary array. As the cycle number increases, different signatures of the deblocking solution's CV curve are observed. Similar to Example 1 above, several signature metrics can be observed on the CV curves to inform on synthesis quality. With synthesis, the absolute oxidation and reduction peak current decrease and the delta V between the peak potentials increases. This indicates evidence of passivation and/or electrode fouling making the electrode less efficient. Furthermore, additional minor oxidation peaks and thicker baselines with increasing synthesis cycles are observed, possibly indicating side products and higher capacitive effects on the electrode. These observed changes may be used for informing on the efficiency of electron transfer to generate the reactant of interest on the primary array.

### Example 6: Evaluation of Cleaning and Pretreatment of Electrodes Pre-Synthesis

The methods described in this disclosure can be used to evaluate the cleanliness of the electrode array before synthesis using the secondary array.

### Experimental Setup

The experimental setup in this example is similar to Example 1. CV curves were generated prior to, and after, cleaning the electrodes of the secondary array.

### Results

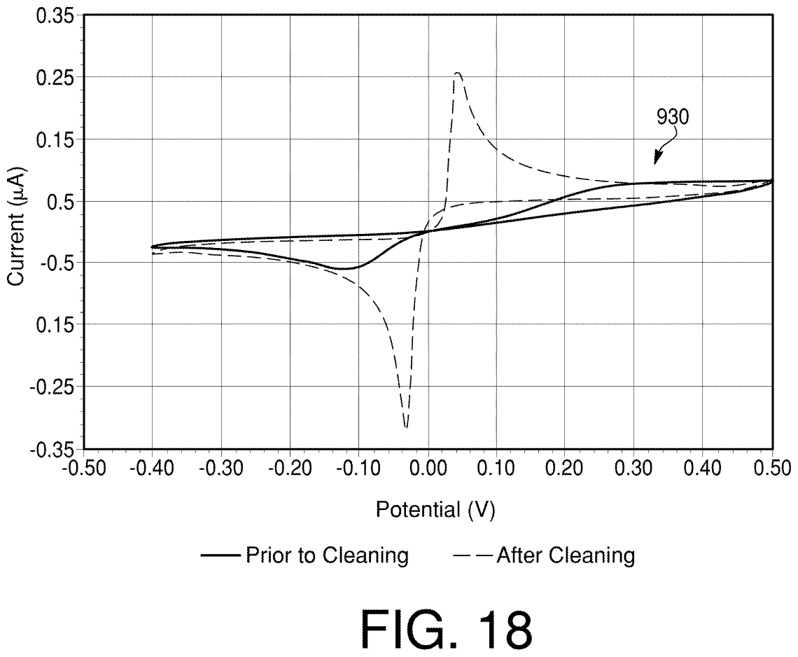

FIG. 18 is an example of using cyclic voltammetry on the secondary inert redox probe solution on the secondary array before and after cleaning. The peak oxidation and reduction currents/voltages illustrate electrode quality before and after cleaning. Before cleaning the electrode, the peak currents are much lower suggesting less charge transferred across the electrode. The peak-to-peak voltage differential is also higher suggesting more energy involved in the charge to transfer pre-cleaning. The data acts as a metric for evaluating a starting substrate for its “cleanliness” and charge transfer efficiency before beginning a synthesis cycle. Pre-cleaning, there are impurities, oxides, adsorbed ions, etc., that can impact charge transfer to/from the electrode to the solution, as seen by the higher oxidation/reduction potentials and lower currents. After cleaning, electrochemistry is improved at the surface and higher currents are obtained at lower applied potentials indicative of a clean surface. This information can be used to verify the electrochemical efficiency of the array before a cyclic synthesis reaction. This tool acts as a quality control metric for the electrochemical chips before synthesis.

### Example 7: Evaluation of Cleaning Methods

As discussed in examples 1-6, cyclic voltammetry or other electroanalytical techniques of the secondary inert redox probe on the secondary array can also be used to compare several cleaning methods and optimize their specific parameters such as incubation times and number of cleaning cycles until the optimal surface is obtained. The metric developed may also act to inform on the best cleaning protocols for each electrochemical chip used.

### Experimental Setup

The experimental set up is similar to the one described in Example 1 above. The electrodes on the secondary array were cleaned using three different cleaning methods: cleaning method 1 included electrochemical cleaning with 0.1M potassium chloride solution, cleaning method 2 included electrochemical cleaning with 0.05M sulfuric acid solution, and cleaning method 3 included chemical cleaning with acetonitrile, followed by incubation for 2 minutes. The negative control did not include any cleaning.

### Results

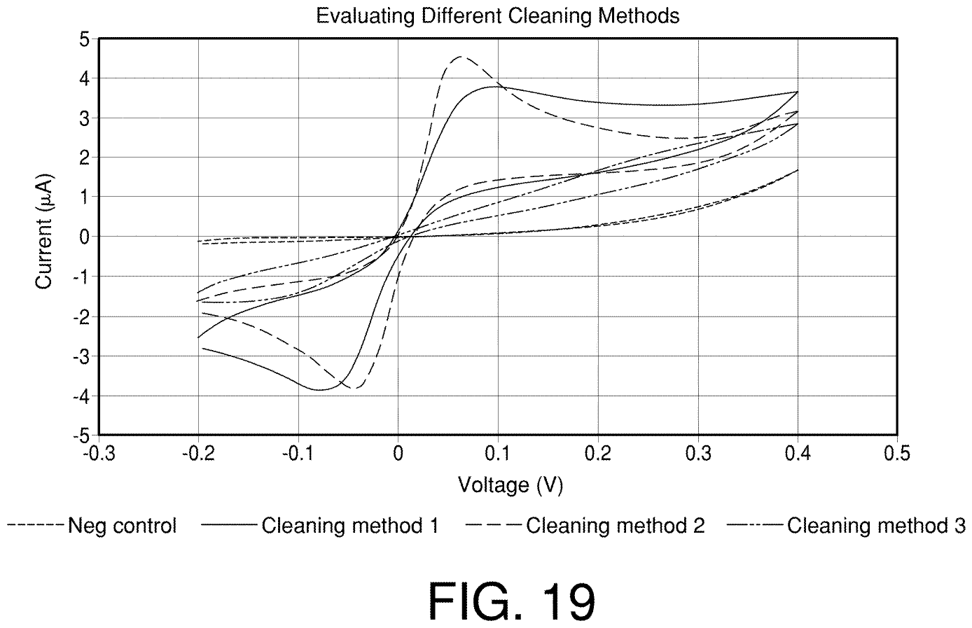

FIG. 19 is an example of using cyclic voltammetry on the secondary inert redox probe solution on the secondary array without cleaning (negative control) and after cleaning with the three different cleaning methods described above. Of the three cleaning methods performed, cleaning method 2 performed the best. The oxidation and reduction peak currents are the highest indicating most charge conducted, and the peak-to-peak voltage differential is minimal indicating optimal energetics for electron transfer.

### Example 8

The secondary array and the methods described herein may be used as a tool for evaluating several electrochemical deblocking cocktails using electroanalytical techniques such as cyclic voltammetry.

### Experimental Setup

The experimental set up is similar to the one described in Example 1 above. The CV performance of three different deblocking solutions on a secondary array, such as the one shown in FIG. 9B, is compared.

### Results

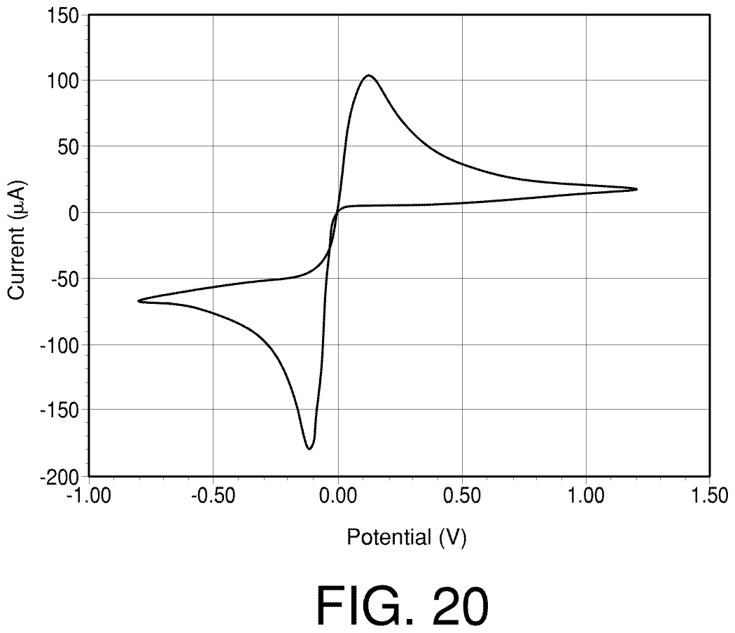

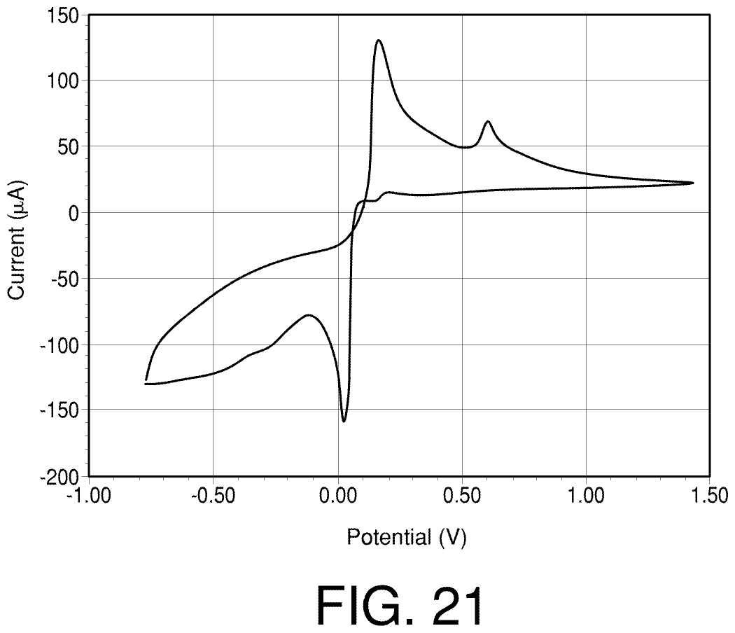

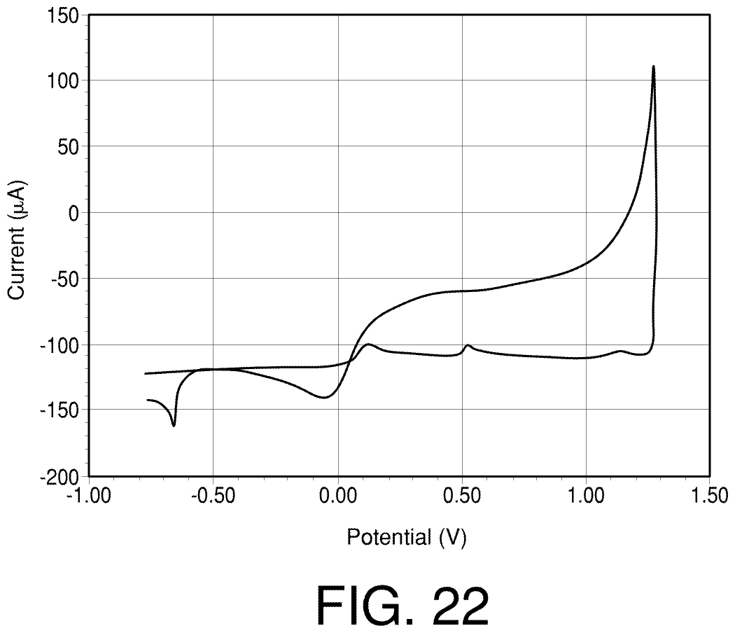

FIGS. 20-22 illustrate the CV curves of different deblocking solutions that may be used for synthesis on the primary array. Information on redox potentials, potential windows, charge transfer and mass transport limited behavior may be obtained for each solution being investigated to help develop better deblocking solutions for electrochemical synthesis. Metrics such as oxidation reduction peak currents and potentials, number of peaks, baseline, side products generation, and reversibility of the redox reaction, may be deduced. These can be used for attaching mechanisms and chemical aspects to the synthesis quality.

### Example 9: Development of a Translational Function

### Experimental Setup

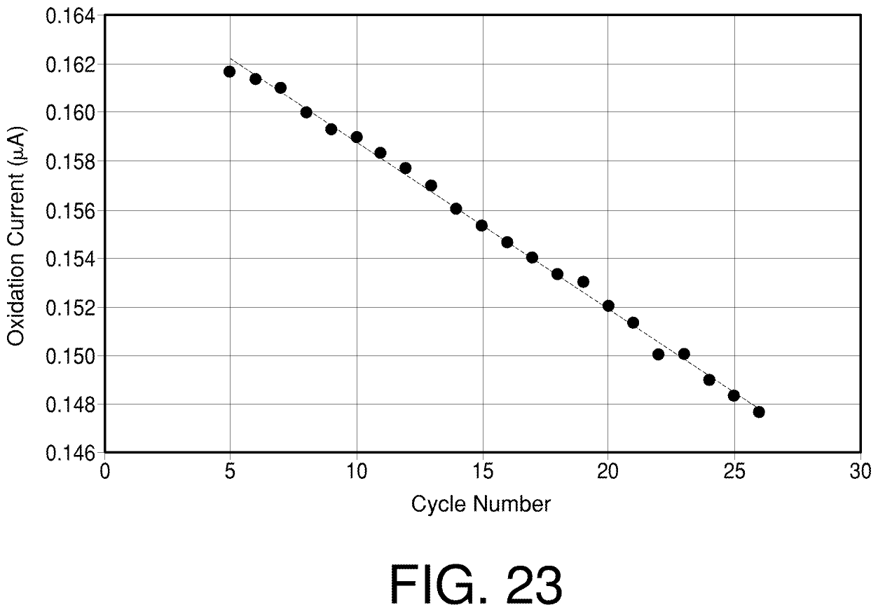

A 1.6V pulse voltage was applied to perform electrochemical deblocking on both the primary and secondary array. This was followed with chronoamperometry of the secondary inert redox probe (e.g., ferrocene) on the secondary array and obtain i vs t plots for a given applied voltage (V_{applied}). This process was repeated for 25 cycles to make the requisite oligomer. Furthermore, the net oxidation current (μA) for ferrocene may be calculated and plotted against the number of cycles to obtain fit type, as show in FIG. 23. The Rₚₐₛₛᵢᵥₐₜᵢₒₙ value was obtained from this curve since Rₚₐₛₛᵢᵥₐₜᵢₒₙ=V_{applied}/slope of the plot.

### Results

As shown in FIG. 23, as the number of cycles progresses the current (i.e., charge transferred) decreases linearly for the same potential applied due to increased passivation of the surface by quinone molecules. Based on electric circuit theory, this increased resistance scales linearly with voltage by Ohm's law and hence the slope of the curve (or the Δi) is used to obtain the ΔV increase that is applied to the primary array during deblocking to get the original current (charge) to be supplied on the primary array for the specific electrochemical deblocking reaction of interest. The graph is used to determine the current decay occurring due to passivation/electrode fouling with synthesis cycles. The point slope equation of the graph in FIG. 23 was calculated to be y=−0.0007x+0.1656. The slope was calculated to be −0.0007. Based on the slope's value, it can be calculated that there is 0.0175 uA of current loss every cycle due to parallel resistance. Taking the intercept value (0.1656) as the original current, a resistance value of 1.6/0.1656=9.66 Mohm is obtained. For making up 0.0175 uA current in the next cycle, with a resistance of 9.66 Mohm, a voltage of 1.769V (1.6V+(0.0175*9.66)) should be applied.

All definitions, as defined and used herein, should be understood to control over dictionary definitions, definitions in documents incorporated by reference, and/or ordinary meanings of the defined terms.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A method of determining a condition on an electrochemical chip comprising a plurality of reaction sites, the method comprising:
conducting a biomolecular synthesis reaction on a primary array of the plurality of reaction sites;
exposing a secondary array of the plurality of reaction sites to a secondary inert redox probe, wherein the secondary inert redox probe provides data on the electrochemical chip condition, and wherein the secondary array is connected to a potentiostat;
receiving, by a computing device, data regarding the secondary array;
comparing, by the computing device, a feature of the data to a pre-determined threshold;
based on the comparison of the feature to the pre-determined threshold, determining, by the computing device, a condition of the electrochemical chip; and
generating, by the computing device, a signal indicative of the condition of the electrochemical chip.

2. The method of claim 1, wherein the method further comprises:
based on the determined condition, altering an electrochemical synthesis parameter of the biomolecular synthesis.

3. The method of claim 1, wherein the method further comprises:
altering an electrochemical synthesis parameter of the biomolecular synthesis based on the determined condition of the electrochemical chip, the electrochemical synthesis parameter of the biomolecular synthesis including at least one of voltage, reactant concentration, or time.

4. The method of claim 1, wherein the data received regarding the secondary array includes an activity or a property of the secondary inert redox probe measured by the potentiostat, and the secondary inert redox probe is ferrocene.

5. The method of claim 1, wherein the data comprises outputs from at least one of cyclic voltammetry, chronoamperometry, chronocoulometry, or electrochemical impedance spectroscopy.

6. The method of claim 1, wherein the biomolecular synthesis comprises oligonucleotide synthesis.

7. The method of claim 1, wherein the method further comprises:
synthesizing biomolecules on the secondary array.

8. The method of claim 1, wherein the data regarding the secondary array is first data, and the method further comprises:
contacting the plurality of reaction sites with a washing solution;
receiving second data regarding the secondary array; and
based on a comparison of the first data to the second data, determining a cleaning state of one or more reaction sites of the plurality of reaction sites.

9. The method of claim 1, wherein determining the condition of the electrochemical chip comprises assessing electrode quality, effectiveness of charge transfer, and/or adsorption.

10. The method of claim 9, wherein assessing electrode quality includes at least one of measuring a thickness of an oxide layer, measuring a fouling of an electrode, measuring a corrosion of an electrode, or measuring an effective electrode area.

11. The method of claim 1, wherein the primary array and the secondary array are exposed to identical electrochemical reactant concentrations.

12. The method of claim 1, wherein the primary array and the secondary array are exposed to substantially the same voltage.

13. A method of determining a condition on an electrochemical chip comprising a plurality of reaction sites, the method comprising:
conducting a biomolecular synthesis reaction on a primary array of the plurality of reaction sites;
connecting a secondary array of the plurality of reaction sites to a potentiostat;
receiving data regarding the secondary array;
comparing a feature of the data to a pre-determined threshold; and
based on the comparison of the feature to the pre-determined threshold, determining a condition of the electrochemical chip.

14. The method of claim 13, wherein the method further comprises:
altering an electrochemical synthesis parameter of the biomolecular synthesis reaction.

15. The method of claim 13, wherein the method further comprises:
altering an electrochemical synthesis parameter of the biomolecular synthesis reaction, the electrochemical synthesis parameter of the biomolecular synthesis reaction including at least one of voltage, reactant concentration, depassivation, electrode cleaning, or time.

16. The method of claim 13, wherein the data comprises outputs from at least one of cyclic voltammetry, chronoamperometry, chronocoulometry, or electrochemical impedance spectroscopy.

17. The method of claim 13, wherein the biomolecular synthesis comprises oligonucleotide synthesis.

18. The method of claim 13, wherein the method further comprises:
synthesizing biomolecules on the secondary array.

19. The method of claim 13, wherein determining the condition of the electrochemical chip comprises at least one of assessing electrode quality, effectiveness of charge transfer, or adsorption.

20. The method of claim 13, wherein the secondary array is located on a lid of the electrochemical chip.

21. The method of claim 13, wherein the secondary array is exposed to a primary deblocking solution.

22. The method of claim 13, wherein the method is further used to select a primary deblocking solution for use during an electrochemical reaction, the method further comprising:
contacting the plurality of reaction sites with a first primary deblocking solution;
receiving first data regarding the secondary array;
contacting the plurality of reaction sites with a second primary deblocking solution;
receiving second data regarding the secondary array;
comparing a feature of the first data to a feature of the second data; and
selecting a primary deblocking solution based on the comparison of the feature of the first data to the feature of the second data.

23. The method of claim 22, wherein the contacting, receiving, and comparing may be repeated for two or more primary deblocking solutions.

24. The method of claim 13, wherein the condition of the electrochemical chip is determined during biomolecular synthesis.

25. The method of claim 13, wherein the condition of the electrochemical chip is determined before biomolecular synthesis.

26. The method of claim 13, wherein the condition of the electrochemical chip is determined after biomolecular synthesis.

27. A system for monitoring electrochemical synthesis, comprising:
an electrochemical chip comprising a primary array configured for biomolecular synthesis and a secondary array configured for electrochemical monitoring, each of the primary array and the secondary array configured to receive the same chemical reagents, and the secondary array configured to be exposed to a secondary inert redox probe; and
a computing device communicatively coupled to the electrochemical chip, the computing device configured to:
receive data including a property of the secondary inert redox probe;
compare the data to a pre-determined threshold;
based on the comparison, determine a condition of the electrochemical chip; and
generate a signal indicative of the determined condition.

28. The system of claim 27, wherein the computing device is further configured to adjust at least one synthesis parameter of the biomolecular synthesis.

29. The system of claim 28, wherein the synthesis parameter includes at least one of voltage, reagent concentration, depassivation, electrode cleaning, or time.

30. The system of claim 27, wherein the condition includes assessing at least one of an electrode quality, an effectiveness of charge transfer, or electrochemistry at an electrode-electrolyte interface.

31. The system of claim 27, wherein the property of the secondary inert redox probe is measured by a potentiostat.

32. The system of claim 27, wherein the primary array and the secondary array are exposed to substantially the same voltage.

33. The system of claim 27, wherein the computing device is further configured to: evaluate a condition of one or more electrodes of the electrochemical chip for reuse after cleavage of a biomolecular synthesized product.

34. The system of claim 27, wherein the computing device is further configured to: evaluate cleanliness of an active area of the electrochemical chip prior to synthesis.

35. The system of claim 27, wherein:
the data regarding the property of the secondary inert redox probe is a first data,
the primary array and the secondary array are configured to be contacted with a washing solution, and
the computing device is further configured to:
receive second data regarding the secondary array;
compare the first data to the second data; and
determine a cleaning state of one or more reaction sites of the primary array based on the comparison; and
generate a signal indicative of the determined cleaning state.
